SEQUENCE LISTING (9 sequences)

SEQ ID NO: 1 - protein, 1014 aa, Escherichia coli
MGNQWQQKYLLEYNELVSNFPSPERVVSDYIKNCFKTDLPWFSRIDPDNAYFICFSQNRSNSRSYTGWDHLGKYKTEVLTLTQAALINIGYRFDVFDDANSSTGIYKTKSADVFNEENEEKMLPSEYLHFLQKCDFAGVYGKTLSDYWSKYYDKFKLLLKNYYISSALYLYKNGELDEREYNFSMNALNRSDNISLLFFDIYGYYASDIFVAKNNDKVMLFIPGAKKPFLFKKNIADLRLTLKELIKDSDKQQLLSQHFSLYSRQDGVSYAGVNSVLHAIENDGNFNESYFLYSNKTLSNKDVFDAIAISVKKRSFSDGDIVIKSNSEAQRDYALTILQTILSMTPIFDIVVPEVSVPLGLGIITSSMGISFDQLINGDTYEERRSAIPGLATNAVLLGLSFAIPLLISKAGINQEVLSSVINNEGRTLNETNIDIFLKEYGIAEDSISSTNLLDVKLKSSGQHVNIVKLSDEDNQIVAVKGSSLSGIYYEVDIETGYEILSRRIYRTEYNNEILWTRGGGLKGGQPFDFESLNIPVFFKDEPYSAVTGSPLSFINDDSSLLYPDTNPKLPQPTSEMDIVNYVKGSGSFGDRFVTLMRGATEEEAWNIASYHTAGGSTEELHEILLGQGPQSSLGFTEYTSNVNSADAASRRHFLVVIKVHVKYITNNNVSYVNHWAIPDEAPVEVLAVVDRRFNFPEPSTPPDISTIRKLLSLRYFKESIESTSKSNFQKLSRGNIDVLKGRGSISSTRQRAIYPYFEAANADEQQPLFFYIKKDRFDNHGYDQYFYDNTVGLNGIPTLNTYTGEIPSDSSSLGSTYWKKYNLTNETSIIRVSNSARGANGIKIALEEVQEGKPVIITSGNLSGCTTIVARKEGYIYKVHTGTTKSLAGFTSTTGVKKAVEVLELLTKEPIPRVEGIMSNDFLVDYLSENFEDSLITYSSSEKKPDSQITIIRDNVSVFPYFLDNIPEHGFGTSATVLVRVDGNVVVRSLSESYSLNADASEISVLKVFSKKF

SEQ ID NO: 2 - protein, 1014 aa, Escherichia coli
MNVQWQQKYLLEYNELVSNFPSPERVVSDYIRRCFKTDLPWFSQVDPDNTYFIRFSQSRSNSRSYTGWDHLGKYKTGVLTLTQAALINIGYHFDVFDDANASAGIYKTSSADMFNEKNEEKMLPSEYLYFLKGCDFSGIYGRFLSDYWSKYYDKFKLLLKNYYISSALYLYKNGEIDEYEYNFSISALNRRDNISLFFFDIYGYYSSDMFVAKNNERVMLFIPGAKKPFLFEKNIADLRISLKNLIKENDNKQLLSQHFSLYSRQDGITYAGVNSVLNAIENDGVFNESYFLYSNKRINNKDVFDAVAFSVKKRSFSDGDIVIKSNSEAQRDYALTILQTILSMTPIFDVAIPEVSVTLGLGIIASSMGISFDQLINGDTYEERRSAIPGLATNAALLGLSFAIPFLISKAGTNQKILSRYTKHEIRTLNETNIDMFLEEYGINKNSISETKVLEVELKGSGQHVNIVKLSDEDNKIVAVKGNSLSGIYYEVDIETGYEISSRRIYRTEYNDKIFWTRGGGLKGGQSFDFESLKLPIFFKDEPYSAVPGSSLSFINDDSSLLYPNSTPKLPQPTPEMEIVNYVKRAGNFGERLVTLMRGTTEEEAWNIARYHTAGGSTEELHEILLGQGPQSSLGFTEYTSNINSADAASRRHFLVVIKVQVKYINNNNVSHVNHWAIPDEAPVEVLAVVDRRFNFPEPSTPPNISIIHKLLSLRYFKENIESTSRLNLQKLNRGNIDIFKGRGSISSTRQRAIYPYFESANADEQQPVFFYIKKNRFDDFGYDQYFYNSTVGLNGIPTLNTYTGEILSDASSLGSTYWKKYNLTNETSIIRVSNSARGANGIKIALEEVQEGKPVIITSGNLSGCTTIVARKGGYLYKVHTGTTIPLAGFTSTTGVKKAVEVFELLTNNPMPRVEGVMNNDFLVNYLAESFDESLITYSSSEQKIGSKITISRDNVSTFPYFLDNIPEKGFGTSVTILVRVDGNVIVKSLSESYSLNVENSNISVLHVFSKDF

SEQ ID NO: 3 - protein, 1014 aa, Yersinia pseudotuberculosis
MKNQWQHQYFLSYSELVANFPSPEKVVSDYIKHKFSTTLPWFGWADPDNLYFIRFTQSRSNNKSYTGWDHLGKYAIETLTLTQAAIVNIGSRFDIFDEANSTAGIYKTNNADSFDETNEAKMLPSEYLYFLRDCDFSNLYNKALSDYWAENYEKFSTLLQNYYISSAYYLYKDSAISKDEYEFSIDAIFNKKSKILRYYFDVYGYYSSDMFVAMNDNKTMLFIPGATNPFIFADNITDLRDKIKALISDKNTRELFSKHFSLYDRQDGNTYLGVNSMLEQIVSGVVDTNYIMYSNKNIRERNVFGSMAFSTRERSFNDGDVIIKSNAEVQRDYALNVLQTILSLSPIFDIVLPEVSIPISLGITASSVGISFDELINGDTYEERRSAIPGLATNTVLLGISFAIPFLISKAEENKLIINNLVGSDENILNKNNLGDFLEKYNISESDIPENGSLVINLKNTNVPVRLVKLNDEEGEIVAIKGSTLSGIYYEVDTETGYEILSRRVFRTEYNEKIYWTRGGGLKGGQPFNFEGLDIPVYFIDKPYSELASSVELSFVNDDSPLLFPEMDSRLPKPTPELDIKYYSSNLSSFKEDTVILMRGTTEEEAWNIANYKTAGGSNKDLEENFIEAGPQFNLSFSEYTSSINSADTASRKHFLVIIKVQVKYISNDNVLYANHWAIPDEAPVEVLAVVDRRFIFPEPPVKPKLSFIQKIANRFLTENVAEISSINFRRLNSGNINVLKGRGVFSSRRLREIYLRFDAANADELRPGDVYVKKTKFDSMGYDSHFYNEGIGINGAPTLNTYTGEYVADSSSQGATYWLKYNLTNETSIIKVSNSARGANGIKIALEEIEENKPVVITSGTLTGCTVVFARKGEYFYAVHTGNSESLIGFTSTSGVAKAIEVLSSLSELEVPALPDVINNNTLVEYLSDNFDSALISYSSSSLKPNSMINISRENVSTFSYYTDDIQLPSFGTSVTILVRTNDNTVVRSLSESYTMNSNSSKMVVFNVLQKDF

SEQ ID NO: 4 - protein, 1451 aa, Bordetella pertussis
MALVGYDGVVEELLALPSEESGDLAGGRAKREKAEFALFSEAPNGDEPIGQDARTWFYFPKYRPVAVSNLKKMQVAIRARLEPESLILQWLIALDVYLGVLIAALSRTVISDLVFEYVKARYEIYYLLNRVPHPLATAYLKRRRQRPVDRSGRLGSVFEHPLWFAYDELAGTVDLDADIYEQALAESIERRMDGEPDDGSLDTAEHDVWRLCRDGINRGEQAIFQASGPYGVVADAGYMRTVADLAYADALADCLHAQLRIRAQGSVDSPGDEMPRKLDAWEIAKFHLAATQQARVDLLEAAFALDYAALRDVRVYGDYRNALALRFIKREALRLLGARRGNASTMPAVAAGEYDEIVASGAANDAAYVSMAAALIAGVLCDLESAQRTLPVVLARFRPLGVLARFRRLEQETAGMLLGDQEPEPRGFISFTDFRDSDAFASYAEYAAQFNDYIDQYSILEAQRLARILALGSRMTVDQWCLPLQKVRHYKVLTSQPGLIARGIENHNRGIEYCLGRPPLTDLPGLFTMFQLHDSSWLLVSNINGELWSDVLANAEVMQNPTLAALAEPQGRFRTGRRTGGWFLGGPATEGPSLRDNYLLKLRQSNPGLDVKKCWYFGYRQEYRLPAGALGVPLFAVSVALRHSLDDLAAHAKSALYKPSEWQKFAFWIVPFYREIFFSTQDRSYRVDVGSIVFDSISLLASVFSIGGKLGSFTRTQYGNLRNFVVRQRIAGLSGQRLWRSVLKELPALIGASGLRLSRSLLVDLYEIFEPVPIRRLVAGFVSATTVGGRNQAFLRQAFSAASSSAGRTGGQLASEWRMAGVDATGLVESTSGGRFEGIYTRGLGPLSECTEHFIVESGNAYRVIWDAYTHGWRVVNGRLPPRLTYTVPVRLNGQGHWETHLDVPGRGGAPEIFGRIRTRNLVALAAEQAAPMRRLLNQARRVALRHIDTCRSRLALPRAESDMDAAIRIFFGEPDAGLRQRIGRRLQEVRAYIGDLSPVNDVLYRAGYDLDDVATLFNAVDRNTSLGRQARMELYLDAIVDLHARLGYENARFVDLMAFHLLSLGHAATASEVVEAVSPRLLGNVFDISNVAQLERGIGNPASTGLFVMLGAYSESSPAIFQSFVNDIFPAWRQASGGGPLVWNFGPAAISPTRLDYANTDIGLLNHGDISPLRARPPLGGRRDIDLPPGLDISFVRYDRPVRMSAPRALDASVFRPVDGPVHGYIQSWTGAEIEYAYGAPAAAREVMLTDNVRIISIENGDEGAIGVRVRLDTVPVATPLILTGGSLSGCTTMVGVKEGYLAFYHTGKSTELGDWATAREGVQALYQAHLAMGYAPISIPAPMRNDDLVSIAATYDRAVIAYLGKDVPGGGSTRITRHDEGAGSVVSFDYNAAVQASAVPRLGQVYVLISNDGQGARAVLLAEDLAWAGSGSALDVLNERLVTLFPAPV

SEQ ID NO: 5 - protein, 240 aa, Salmonella typhimurium
MTKITLSPQNFRIQKQETTLLKEKSTEKNSLAKSILAVKNHFIELRSKLSERFISHKNTESSATHFHRGSASEGRAVLTNKVVKDFMLQTLNDIDIRGSASKDPAYASQTREAILSAVYSKNKDQCCNLLISKGINIAPFLQEIGEAAKNAGLPGTTKNDVFTPSGAGANPFITPLISSANSKYPRMFINQHQQASFKIYAEKIIMTEVAPLFNECAMPTPQQFQLILENIANKYIQYTP

SEQ ID NO: 6 - protein, 240 aa, Salmonella typhimurium
MTNITLSTQHYRIHRSDVEPVKEKTTEKDIFAKSITAVRNSFISLSTSLSDRFSLHQQTDIPTTHFHRGNASEGRAVLTSKTVKDFMLQKLNSLDIKGNASKDPAYARQTCEAILSAVYSNNKDQCCKLLISKGVSITPFLKEIGEAAQNAGLPGEIKNGVFTPGGAGANPFVVPLIASASIKYPHMFINHNQQVSFKAYAEKIVMKEVTPLFNKGTMPTPQQFQLTIENIANKYLQNAS

SEQ ID NO: 7 - protein, 166 aa, Shigella flexneri
MEIQNTKSAPILYTDISTKQTQSSSETQKSQNYQQLAAHIPLNVGKNPVLTTTLNDDQLLKLSEQVQHDSEIIARLTDKKMKDLSEMSHTITPENTLDISSLSSNAVSLIISVAVLLSALRTAETRLGSQLSLIAFDATKSAAENIVRQGLAALSSSITGAVTQVG

SEQ ID NO: 8 - protein, 1186 aa, Helicobacter pylori
MTNETIDQTRTPDQTQSQTAFDPQQFINNLQVAFIKVDNVVASFDPDQKPIVDKNDRDNRQAFDGISQLREEYSNKAIKNPTKKNQYFSDFIDKSNDLINKDNLIDVESSTKSFQKFGDQRYQIFTSWVSHQKDPSKINTRSIRNFMENIIQPPIPDDKEKAEFLKSAKQSFAGIIIGNQIRTDQKFMGVFDESLKERQEAEKNGGPTGGDWLDIFLSFIFNKKQSSDVKEAINQEPVPHVQPDIATTTTDIQGLPPEARDLLDERGNFSKFTLGDMEMLDVEGVADIDPNYKFNQLLIHNNALSSVLMGSHNGIEPEKVSLLYAGNGGFGDKHDWNATVGYKDQQGNNVATLINVHMKNGSGLVIAGGEKGINNPSFYLYKEDQLTGSQRALSQEEIRNKVDFMEFLAQNNTKLDNLSEKEKEKFQNEIEDFQKDSKAYLDALGNDRIAFVSKKDTKHSALITEFNNGDLSYTLKDYGKKADKALDREKNVTLQGSLKHDGVMFVDYSNFKYTNASKNPNKGVGATNGVSHLEAGFNKVAVFNLPDLNNLAITSFVRRNLENKLTAKGLSLQEANKLIKDFLSSNKELAGKALNFNKAVAEAKSTGNYDEVKKAQKDLEKSLRKREHLEKEVEKKLESKSGNKNKMEAKAQANSQKDEIFALINKEANRDARAIAYTQNLKGIKRELSDKLEKISKDLKDFSKSFDEFKNGKNKDFSKAEETLKALKGSVKDLGINPEWISKVENLNAALNEFKNGKNKDFSKVTQAKSDLENSVKDVIINQKVTDKVDNLNQAVSVAKAMGDFSRVEQVLADLKNFSKEQLAQQAQKNEDFNTGKNSELYQSVKNSVNKTLVGNGLSGIEATALAKNFSDIKKELNEKFKNFNNNNNGLKNSTEPIYAKVNKKKTGQVASPEEPIYTQVAKKVNAKIDRLNQIASGLGGVGQAAGFPLKRHDKVDDLSKVGLSASPEPIYATIDDLGGPFPLKRHDKVDDLSKVGRSRNQELAQKIDNLNQAVSEAKAGFFGNLEQTIDKLKDSTKKNVMNLYVESAKKVPASLSAKLDNYAINSHTRINSNIQNGAINEKATGMLTQKNPEWLKLVNDKIVAHNVGSVSLSEYDKIGFNQKNMKDYSDSFKFSTKLNNAVKDIKSGFTHFLANAFSTGYYCLARENAEHGIKNVNTKGGFQKS

SEQ ID NO: 9 - protein, 201 aa, Homo sapiens
SSGPSSSLDNGNSLDVLKNHVLNELIQTERVYVRELYTVLLGYRAEMDNPEMFDLMPPLLRNKKDILFGNMAEIYEFHNDIFLSSLENCAHAPERVGPCFLERKDDFQMYAKYCQNKPRSETIWRKYSECAFFQECQRKLKHRLRLDSYLLKPVQRITKYQLLLKELLKYSKDCEGSALLKKALDAMLDLLKSVNDSMHQI

(19) United States
(12) Patent Application Publication
(10) Pub. No.: US 2010/0266632 A1
(43) Pub. Date: Oct. 21, 2010

(54) VACCINE COMPOSITION COMPRISING AN IMMUNOADJUVANT COMPOUND CONSISTING OF A RHO GTPASE FAMILY ACTIVATOR

(76) Inventors: Emmanuel Lemichez, Nice (FR); Cecil Czerkinsky, Nice (FR); Fabienne Anjuere, Nice (FR); Patrice Boquet, Villefranche-Sur-Mer (FR); Patrick Munro, Nice (FR); Gilles Flatau, Nice (FR)

Correspondence Address: YOUNG & THOMPSON, 209 Madison Street, Suite 500, Alexandria, VA, 22314, US

(21) Appl. No.: 12/627,575

(22) Filed: Nov. 30, 2009

Related U.S. Application Data
(63) Continuation-in-part of application No. 10/589,505, filed on Mar. 8, 2007, now Pat. No. 7,655,240.

(30) Foreign Application Priority Data
Feb. 26, 2004 (EP) 04300100.7

(51) Int. Cl. A61K 39/39; A61K 39/02; A61K 39/12; A61K 39/00; A61K 39/106; A61K 39/108; A61K 39/08; A61K 39/21; A61P 33/00; A61P 31/18; A61P 31/12; A61P 31/10; A61P 31/04

(52) U.S. Cl. 424/208.1; 424/278.1; 424/184.1; 424/234.1; 424/204.1; 424/274.1; 424/265.1; 424/261.1; 424/257.1; 424/247.1

(57) ABSTRACT

An immunogenic or vaccine composition comprising an immunoadjuvant compound consisting of a Rho GTPase activator. The Activators of Rho GTPases, namely the cytotoxic necrotizing factor 1 (CNF1), and DNT bear immunostimulatory properties towards the systemic response to orally administered ovalbumine.

### CROSS REFERENCE TO RELATED APPLICATIONS

This application is a Continuation-in-Part of copending application Ser. No. 10/589,505 filed on Aug. 15, 2006; which is the 35 U.S.C. 371 national stage of international application PCT/EP2005/002105 filed on Feb. 25, 2005, which claimed priority of EP Application No.: 04300100.7 filed on Feb. 26, 2004. The entire contents of each of the above-identified applications are hereby incorporated by reference.

### FIELD OF THE INVENTION

The present invention relates to a vaccine composition or an immunogenic composition comprising an immuno adjuvant compound, wherein said immuno adjuvant compound consists of a RHO GTPase family activator.

### BACKGROUND OF THE INVENTION

Vaccines have proven to be successful, highly acceptable methods for the prevention of infectious diseases. There are cost effective, and do not induce antibiotic resistance to the target pathogen or affect normal flora present in the host. In many cases, such as when inducing anti-viral immunity, vaccines can prevent a disease for which there are no viable curative or ameliorative treatments available.

Vaccines function by triggering the immune system to induce a response to an agent, or an antigen, typically in an infectious organism or a portion thereof that is introduced into the body in a non-infectious or non-pathogenic form.

Once the immune system has been “primed” or sensitised to the organism, later exposure of the immune system to this organism, results in a rapid and robust immune response that destroys the pathogen before it can multiply or infect enough cells in the host organism to cause disease symptoms.

The agent, or antigen, used to prime the immune system can be the entire organism in a less infectious state, known as an attenuated organism, or in some cases, component of the organism such as carbohydrate proteins or peptides representing various structural components of the organism.

In many cases, it is necessary to enhance the immune response to the antigens present in a vaccine in order to stimulate the immune system to a sufficient extent to make a vaccine effective, i.e., to confer immunity. Many proteins and most peptide and carbohydrate antigens, administered alone, do not elicit a sufficient antibody response to confer immunity. Such antigens need to be presented to the immune system in such a way that they will be recognized as foreign and will elicit an immune response.

To this end, additives like adjuvants, have been devised, which immobilise antigens and stimulate the immune response.

Recombinant proteins are promising vaccine or immunogenic composition candidates because they can be produced at high yield and purity and manipulated to maximize desirable activities and minimize undesirable ones.

However, because they can be poorly immunogenic, methods to enhance the immune response to recombinant proteins are important in the development of vaccines or immunogenic compositions. Such antigens, especially when recombinantly produced, may elicit a stronger response when administrated in conjunction with an adjuvant.

The best known adjuvant, Freund's complete adjuvant, consists of a mixture of mycobacteria in an oil/water emulsion.

Freund's adjuvant works in two ways; first, by enhancing cell and humoral-mediated immunity, and second by blocking rapid dispersal of the antigens challenge, also called “depot effect”. However, due to frequent toxic physiological and immunological reactions to this material, Freund's adjuvant cannot be used in humans.

Another molecule that has been shown to have stimulatory or adjuvant activity is endotoxin, although known as lipopolysaccharide (LPS).

LPS stimulates the immune system by triggering an immediate immune response, a response that has evolved to enable an organism to recognize endotoxin and the invading bacteria (of which it is a component) without the need for the organism to have been previously exposed. But LPS is although too toxic to be a viable adjuvant.

Thus, there is a recognized and permanent need in the art for new compounds which can be administered with antigens in order to stimulate the immune system and generate a more robust antibody response to the antigen than will be seen if the antigens were injected alone.

Additionally, it should be noted that parenteral administration i.e. intramuscularly or sub-cutaneous, of antigens of vaccines are normally regarded as the most convenient way of administration.

However, the injection presents a range of disadvantages. It requires the use of sterile syringes and may cause pains and irritations, particularly in the case of repeated injections, including the risk of infection. More significantly, intramuscularly injections are often poorly tolerated. There is often likely to be indurations (hardening of tissue) haemorrhages and/or necrosis (local death of tissue) at the injection site. Besides, untrained person cannot administer injections.

Based on these observations, it should be noted that mucosal immunity has take a considerable importance in vaccine development because nearly all viral, bacterial and parasitic agent that cause disease of the intestinal, respiratory and genital tracks enter through the mucosal barrier. Furthermore, mucosal and systemic immune responses are often elicited and regulated independently, and induction of protective immunity at the most frequent sites of entry is likely to be most effective. Additionally, young children and elderly individuals may respond better to mucosal vaccines because the mucosal immune system develops earlier and appears to remain functional longer than the systemic compartment. Mucosal immunisations are also easier and less expensive than systemic immunisations. For example, the existence of an oral polio vaccine has allowed immunisation campaigns that may soon eradicate polio worldwide.

Accordingly, it is also an object of the present invention to provide a vaccine composition comprising an immunoadjuvant compound which could be administered by the mucosal route. These and further objects will be apparent to one ordinary skill in the art.

### SUMMARY OF THE INVENTION

The present invention is based on the experimental findings that an activator of Rho GTPases, namely the cytotoxic necrotizing factor 1 (cnf1) bears immunostimulatory properties towards the systemic and mucosal responses to orally administered ovalbumine, a prototype soluble protein antigen. CNF1 consists of an injection domain (amino acid residues 1-719 of SEQ ID NO:1), allowing the binding and endosomal penetration of the toxin, followed by the intracytoplasmic injection of its catalytic domain (amino acid residues 720-1014 of SEQ ID NO:1), responsible for Rho GTPases protein family activation.

A first object of the invention consists in a vaccine or an immunogenic composition comprising an immunoadjuvant compound, wherein said immunoadjuvant compound consists of a Rho GTPase activator.

More precisely, the present invention relates to an immunogenic composition comprising:
one or more antigens against which an immune response is sought, and
an immunoadjuvant which consists of a Rho GTPase activator which is able to maintain a Rho GTPase protein in a form bound to GTP;
   wherein the said immunoadjuvant is able to enhance the immune response against the said one or more antigens and wherein the said immunoadjuvant is distinct from anyone of the said one or more antigens.

In some embodiments, the said immunogenic composition consists of a vaccine composition.

In another aspect, the invention relates to a vaccine or an immunogenic composition wherein said immunoadjuvant compound is selected from the group consisting of:
a polypeptide comprising the amino acid sequence starting at the amino acid residue 720 and ending at the amino acid residue 1014 of sequence SEQ ID NO:1,
a polypeptide comprising the amino acid sequence starting at the amino acid residue 720 and ending at the amino acid residue 1014 of sequence SEQ ID NO:2,
a polypeptide comprising the amino acid sequence starting at the amino acid residue 720 and ending at the amino acid residue 1014 of sequence SEQ ID NO:3,
a polypeptide comprising the amino acid sequence starting at the amino acid residue 1146 and ending at the amino acid residue 1451 of sequence SEQ ID NO:4,
a polypeptide comprising the amino acid sequence SEQ ID NO:5,
a polypeptide comprising the amino acid sequence SEQ ID NO:6,
a polypeptide comprising the amino acid sequence SEQ ID NO:7,
a polypeptide comprising the amino acid sequence SEQ ID NO:8, and
a polypeptide comprising the amino acid sequence SEQ ID NO:9.

The present invention also relates to a vaccine or an immunogenic composition wherein the immunoadjuvant compound is a protein comprising a polypeptide consisting of; from the N-terminal end to the C-terminal end, respectively:
a) the injection domain of a Rho GTPase activator, and
b) the catalytic domain of a Rho GTPase activator.
   Another object of the invention is a method for inducing an immune response in a patient in need, the said method comprising the steps of:
(i) providing an immunogenic composition as above-defined
(ii) administering the immunogenic composition provided in step (i) to a patient in need thereof
   A further object of the invention is a method for preparing an immunogenic composition able to induce an immune response to one or more antigens, the said method comprising:
(i) providing one or more antigens against which an immune response is sought
(ii) providing an immunoadjuvant as above-defined and
(iii) mixing the said one or more antigens from step (i) and the said immunoadjuvant from step (ii) optionally in the presence of one or more pharmaceutically acceptable excipients

### DESCRIPTION OF DRAWINGS

FIG. 1: CNF1 Effects on Cell Signaling Pathway.

1A: Immunoblots showing the kinetics of CNF1-induced activation of Rho, Rac and Cdc42 in contrast to Ras, in HUVEC. Cells were treated with 10⁻⁹M CNF1 for different periods of time. Cell lysates were subjected to GST-fusion protein pull-down assays (noted GTPases-GTP). In parallel, 2% of each cell lysate were processed for immunoblotting to monitor their cellular depletion (noted Total-GTPases).

1B: Quantification of the CNF1-induced Rho protein activation. Immunoblots were scanned and quantified using N.I.H. Image 1.6. The level of activated Rho proteins was compared to the total Rho GTPase level present in 2% of control cell lysates (mean value of three independent experiments±SD).

1C: Immunoblots showing the interference of native CNF1 and catalytic inactive CNF1-C866S on cell signaling. HUVEC were treated with “10⁻⁹M” CNF1 or CNF1-C866S for the indicated periods of time, prior to immunoblotting analysis. MAP kinase signaling was investigated using anti-phosphop44/42 MAP Kinase (noted P-p44/42) and anti-phospho-p38 MAP Kinase (noted P-p38) antibodies. Jun kinase activity was investigated by anti-phospho-c-jun (noted P-c-jun) immunoblotting. NF-kappaB signaling pathway activation was investigated by following the IkBα cellular depletion on immunoblots.

FIG. 2: Catalytic Active CNF1 Stimulates Serum IgG Responses to Orally Administered Ovalbumin (OVA).

Five groups of mice were fed OVA alone (control) or co-administered with either CNF1 (1 or 10 μg) or CNF1-C866S (10 μg) or CT (10 μg). Groups of eight mice were immunized with CNF1 or CNF1-C866S, whereas groups of four mice were immunized with OVA alone or OVA+CT. Groups of mice were challenged once, 2 weeks after the first immunization and sera collected 30 days after the first immunization. Levels of the seric anti-OVA IgG titers are expressed as geometric means (histogram and mean values) of the total IgG titers. These results are representative of two independent experiments. Anti-OVA IgG titers from individual animals are displayed (•).

FIG. 3: DNT Catalytic Domain Stimulates Serum IgG Responses to Orally Administered Ovalbumin (OVA).

3A: Immunoblots showing the kinetics of DNT-Cdinduced activation and cellular depletion of Rac. 804G cells were treated with 100 μg of DNT-CD and processed for activated Rac measurements by GST-Pak pull-down (noted RacGTP). Immunoblotting of 10 μg of total lysate was performed to visualize DNT-CD induced Rac depletion (noted Rac) and equal quantities of proteins engaged in the GST pull-down (actin).

3B: Comparison of the cellular activities of CNF-CD and DNT-CD. The graph illustrates the percentage of HEp-2 multinucleated cells measured 48 h following intoxication by different concentrations of either CNF-CD or DNT-CD.

3C: Serum IgG responses to orally administered ovalbumin (OVA). Three groups of 4 mice were fed with OVA alone (control) or co-administered with either CNF-CD (100 μg) or DNT-CD (100 μg). Groups of mice were challenged twice, 2 and 5 weeks after the first immunization and sera collected 30 and 60 days after the first immunization. Levels of the seric anti-OVA IgG titers are expressed as geometric means of the total IgG titers.

FIG. 4: CNF1, CNF1-C866S and CT Induction of Anti-OVA Ig Subclasses.

Three groups of three mice were challenged twice after the first immunization and sera collected 45 days after the first immunization. Levels of the anti-OVA Ig subclasses are expressed as geometric means (histogram).

FIG. 5: CNF1 Induction of Mucosal Anti-OVA IgA Response.

Two groups of three mice were challenged twice, after the first immunization, with OVA supplemented with 10 μg of either CNF1 or CNF1-C866S. Mice were processed according to the PERFEXT method (see the section Material and Method). Levels of the anti-OVA IgA responses are expressed as geometric means (histogram).

FIG. 6: Histology of Small Intestines of Mice Fed CNF1 or CNF1-C866S as Compared to Control Untreated Mice.

Shown are paraffin sections stained with haematoxylin and eosin.

FIG. 7: Measure of the Immunoadjuvant Properties and Toxin Activity of CNF1 and DNT.

7A: Measure of the toxin activity of CNF1, CNF1-CTER (720-1014), DNT-CTER (1154-1451) estimated by HEp-2 cells multinucleation assay, as previously described (Lemichez et al., 1997). As previously reported, CNF1-CTER is poorly active on cells due to its inability to penetrate into the cytosol (Lemichez et al., 1997). DNT-CTER shows a one thousand lower activity, as compared to CNF1.

7B: Serum IgG antibody responses to orally co-administered ovalbumin (OVA) and DNT or CNF1-toxin catalytic domains. Groups of 4 mice were fed OVA alone or co-administered with either CNF1-CTER (720-1014) (100 μg) or DNT-CTER (1154-1451) (100 μg). For CNF1, a group of height mice were fed OVA and CNF1 (10 μg). Mice were challenged once, two weeks after the first immunization and sera collected 30 days after the first immunization. Data are expressed as geometric mean serum IgG anti-OVA Ab titers.

FIG. 8: Secretion of IL-1β by THP1 Cells Transfected with a DNA Construct Encoding for a Constitutively Activated Rho GTPase or an Inactive Rho GTPase.

The expression of an active form of a Rho GTPase in THP1 cell line induces a significant increase of the secretion of IL-1β. In the case of the expression of an inactive Rho GTPase, the level of IL-1β secretion remains unchanged as compared to control cells (THP1 cells transfected with a construct encoding for GFP).

X-coordinate: Rho GTPase encoded by the construct transfected in THP1 cells. RhoA V14, Rac1 L61, Rac2 L61 and Cdc42 L61: activated Rho GTPase mutants. RhoA N19, Rac1 N17, Rac2 N17 and Cdc42 N17: inactive Rho GTPase mutants.

Y-coordinate: concentration of IL-1β in the supernatants of transfected THP1 cells.

### DETAILED DESCRIPTION OF THE INVENTION

The inventors have found according to the invention that Rho GTPase activators bear immunoadjuvant properties in vivo, when co-administered with an antigen, like ovalbumin.

Rho proteins are essential regulatory molecules controlling the actin cytoskeleton organisation and dynamics to accomplish different tasks such as cell polarity, movement, differentiation and phagocytosis (Takai et al., 2001, Etienne-Manneville et al., 2002, Chimini and Chavrier, (2000)). Importance of Rho proteins in physiology is also evidenced by their direct or indirect implication as part of signaling molecules found mutated in human genetic disorders, as well as targets of numerous bacterial virulence factors and toxins (Boettner and Van Aelst, (2002) Boquet and Lemichez, (2003).

Rho proteins interfere with a large variety of signaling pathways controlling gene transcription (Bishop et al., 2000). Among them, a recent report has evidenced the activation of Rac and Cdc42 downstream the Toll-like receptor 2, a gram positive pathogen molecular pattern recognition receptor (PAMP) (Arbibe et al. (2000), Medzhitov et al. (2002).

Also exemplifying the inter-relation between Rho proteins and the host defences is the Rac, Cdc42, VAV and WASP formation of a supra-molecular activation complex (SMAC or “immunological synapse” crucial for lymphocyte activation (Krawczyk et al. 2001).

Many different pathogenic bacteria have evolved virulence factors and toxins aimed at mimicking an activation of Rho GTPase protein family, naturally occurring in eukaryotic cells via specific regulators namely GEF (for guanine nucleotide exchange factors). These cellular GEF consist in domains comprised in large proteins as best described for Dbl (Olson et al., 1996; Schmidt and Hall 2002). Despite their lack of sequence homologies, virulence factors of pathogenic bacteria, for instance SopE and SopE2 from Salmonella have a GEF-like activity (Galan et al., 2000). Some other known factors of pathogenic bacteria, namely IpaC from Shigella and CagA from Helicobacter, activate Rho GTPases by yet uncharacterised molecular mechanisms (Tran Van Nhieu et al., 2000; Boquet and Lemichez 2003). Finally, a group of bacterial toxins comprising CNF1, CNF2, CNFY and DNT also activates Rho proteins through a post-traductional modification (also called post-translational modification). The catalytic sites of these proteins are closely related since the catalytic site of CNF1 (CNF1-CD) is 84% identical to that of CNF2 (CNF2-CD), and 22% identical to that of DNT (DNT-CD) (Boquet and Lemichez 2003).

According to the invention, the inventors have now surprisingly found that the cytotoxic necrotising factor 1 (CNF1) has immunoadjuvant properties. More precisely, the inventors have found that CNF1 bears immunostimulatory properties toward the systemic and mucosal responses to orally administrated ovalbumin in mice.

Additionally, the inventors have found that a mutant of CNF1, namely CNF1-C866S, a catalytically inactive mutant of CNF1 toward GTPases, in contrast to the wild type toxin, does not stimulate the systemic and mucosal responses to ovalbumin. This result points for Rho GTPases proteins activation being directly involved in the immunostimulatory effects of CNF1.

Supporting this point, the inventors have also found according to the invention that the catalytic domain of CNF1, and the catalytic domain of DNT, another Rho GTPase activator, bear also immunoadjuvant properties in vivo, when co-administered with an antigen, like ovalbumin.

The inventors have also shown in vitro that the expression of a constitutively activated Rho GTPase in the THP1 cell line (Human acute monocytic leukemia cell line) induces a significant increase of the secretion of IL1-β which is known to be an effective mucosal and systemic immunoadjuvant. Such an induction of IL-1β is not observed when THP1 cells are transfected with a construct encoding an inactivated Rho GTPase.

Taken together, these results clearly demonstrate that various Rho GTPases activators, structurally distinct, one from the others, have immunoadjuvant properties which result from their functional feature (i.e. their ability to activate Rho GTPase). The said immunoadjuvant properties are thus independent from their structural features. Without wishing to be bound by any theory, the inventors believe that the main mechanism underlying the immunoadjuvant properties of Rho GTPase activators consists of the induction of the IL1-β subsequently to Rho GTPase activation.

Further, a scientific article (Müller, 2009), published after the filing date of application Ser. No. 10/859,505 (called parent application hereafter) from which the present application is a continuation-in-part application, has corroborated the results shown by the inventors. Müller et al. have confirmed that SOPE-2, a Rho GTPase activator described as an immunoadjuvant in the parent application, triggers mucosal inflammation in vivo through the caspase-1 activation-dependent release of mature IL-1β. Müller et al. have also confirmed in vitro that this mechanism is dependent of the activation of Rho GTPases by SOPE-2.

Furthermore, the inventors have found that non neutralizing anti-CNF1 antibodies are naturally found in humans, and that CNF1 activates the Rho GTPase proteins only transiently. Taken together these results demonstrate that CNF1 can be used as an immunoadjuvant compound, deserved of adverse effects such as the toxic effects described for LPS or Cholera Toxin B.

Accordingly, a first object of the invention consists in a vaccine or an immunogenic composition comprising an immunoadjuvant compound, wherein said immunoadjuvant compound consists of a Rho GTPase activator.

By “immunoadjuvant” it is herein intended a substance enhancing the immunogenicity of an antigen.

By “Rho GTPase activator” it is intended herein a compound, which maintains Rho GTPases in a form bound to GTP. In other words, “Rho GTPase activator” encompasses polypeptides which exhibit Rho GTPase-activation ability. It is self-evident that “Rho GTPase activator” does not include proteins derived from Rho GTPase activator which are unable to activate Rho GTPases.

In other words, proteins resulting from the chemical/physical treatment or from the genetic alteration of a Rho GTPase activator are excluded from the scope of the present invention if the said genetic alteration or the said chemical/physical treatment leads to the loss of the GTPase-activation ability. For example, CNF1-C866S is not a “Rho GTPase activator” as defined herein since the said protein fails to activate Rho GTPases. Similarly, toxoids resulting from the heat treatment or the chemical treatment (such as formalin) of Rho GTPase activators do not belong to the group of Rho GTPase activators, as defined herein.

By “Rho GTPases”, the one skilled in the art will understand the proteins belonging to the Rho GTPase family, which encompasses RhoA, RhoB, RhoC, Rac1, Rac2 and Cdc42. (Burridge and Wennerberg, 2004).

The level of Rho GTPase bound to GTP can be easily measured by the methods, referred by those skilled in the art as GST-pull down assays and described for RhoA, B and C by Ren et al., 1999 and for Rac1, Rac2 and Cdc42 by Manser et al., 1998. These methods are described in the section Materials and methods.

Accordingly, in a preferred embodiment, the vaccine or the immunogenic composition comprises an immunoadjuvant which consists of a Rho GTPase activator which is able to maintain a Rho GTPase protein in a form bound to GTP

In some embodiments, the vaccine or the immunogenic composition, as defined above, comprises an immunoadjuvant consisting of a Rho GTPase activator which is able to activate a Rho GTPase through a post-translational modification.

Examples of such Rho GTPase activators are proteins belonging to the bacterial toxin group comprising CNF1, CNF2, CNFY and DNT

In other embodiments, the invention also concerns a vaccine or an immunogenic composition, wherein said immunoadjuvant is selected from the group consisting of:
a polypeptide comprising the amino acid sequence starting at the amino acid residue 720 and ending at the amino acid residue 1014 of sequence SEQ ID NO:1,
a polypeptide comprising the amino acid sequence starting at the amino acid residue 720 and ending at the amino acid residue 1014 of sequence SEQ ID NO:2,
a polypeptide comprising the amino acid sequence starting at the amino acid residue 720 and ending at the amino acid residue 1014 of sequence SEQ ID NO:3,
a polypeptide comprising the amino acid sequence starting at the amino acid residue 1146 and ending at the amino acid residue 1451 of sequence SEQ ID NO:4,
a polypeptide comprising the amino acid sequence SEQ ID NO:5,
a polypeptide comprising the amino acid sequence SEQ ID NO:6,
a polypeptide comprising the amino acid sequence SEQ ID NO:7,
a polypeptide comprising the amino acid sequence SEQ ID NO:8, and
a polypeptide comprising the amino acid sequence SEQ ID NO:9.

A Rho GTPase activator encompasses peptides comprising the amino acid sequence of interest starting at the amino acid residue 720 and ending at the amino acid residue 1014 of sequence SEQ ID NO:1 described above, and comprising a N-terminal amino acid sequence, linked to the amino group of the residue 720 of sequence SEQ ID NO:1.

Preferably, the N-terminal amino acid sequence has a length up to 800 amino acid residues.

Preferably, the N-terminal amino acid sequence is homologous to a part or to the full length amino acid sequence starting at the amino acid residue 1 and ending at the amino acid residue 719 of CNF1 of SEQ ID NO:1.

In such a case, the N-terminal amino acid sequence can comprise substitutions of non-essential amino acid comprised in the sequence starting at the amino acid residue 1 and ending at the amino acid residue 719 of CNF1 of SEQ ID NO:1.

A “non essential” amino acid residue is an amino acid residue that can be altered from the wild type sequence of CNF1 without altering the activating properties of Rho GTPases, whereas an “essential” amino acid residue is required for biological activity.

A Rho GTPase activator encompasses also peptides comprising two or more repeated motifs of the sequence 720-1014 of interest. In such a case, said peptide can comprise also an N-Terminal sequence as defined above.

A Rho GTPase activator encompasses also peptides structurally similar to those described above, derived from the catalytic domain of CNF2 of sequence SEQ ID NO:2, the catalytic domain of CNF of sequence SEQ ID NO:3 and the catalytic domain of DNT of sequence SEQ ID NO:4.

The use of the catalytic domain of Rho GTPase activator, as described above, is of particular interest. Indeed, as demonstrated in example 6, in the case of CNF1, and DNT, the use of the catalytic domain of these proteins is less toxic for cells than the overall proteins, but is sufficient to confer immunoadjuvanticity.

A Rho GTPase activator encompasses also peptides comprising:
the amino acid sequence SEQ ID NO:5 corresponding to SOPE, or
the amino acid sequence SEQ ID NO:6 corresponding to SOPE2, or
The amino acid sequence SEQ ID NO:7 corresponding to IpaC, or
the amino acid sequence SEQ ID NO:8 corresponding to CagA, or
the amino acid sequence SEQ ID NO:9 corresponding to the GEF sequence of Dbl,
   which include more amino acids, and exhibit at least the same activity towards Rho GTPase activation.

Alternatively, the immunoadjuvant according to the invention is selected from the group consisting of:
a polypeptide comprising the amino acid sequence SEQ ID NO:1,
a polypeptide comprising the amino acid sequence SEQ ID NO:2,
a polypeptide comprising the amino acid sequence SEQ ID NO:3, and
a polypeptide comprising the amino acid sequence SEQ ID NO:4.

Another object of the invention consists in a vaccine composition, wherein said immunoadjuvant compound is a protein comprising a polypeptide consisting of; from the N-terminal end to the C-terminal end, respectively:
a) the injection domain of a Rho GTPase activator, and
b) the catalytic domain of a Rho GTPase activator.

By “injection domain of a Rho GTPase activator” it is intended herein, an amino acid sequence allowing the binding and intracellular penetration of a catalytic domain of a Rho GTPase activator.

By “catalytic domain of a Rho GTPase activator” it is intended herein, an amino acid sequence able to activate a Rho GTPase.

The attachment of the injection domain to the catalytic domain above mentioned, to produce a fusion protein may be effected by any means which produces a link between the two constituents, which is sufficiently stable to withstand the conditions used and which does not alter the function of either constituent.

Preferably, the link between them is covalent.

Numerous chemical cross-linking methods are known and potentially applicable for producing the fusion protein. For example, non-specific chemical cross-linking methods, or preferably methods of direct chemical coupling to a functional group, found only once or a few times in one or both of the polypeptides to be cross-linked.

Coupling of the two constituents can also be accomplished via a coupling or conjugating agent. There are several intermolecular cross-linking reagents, which can be used (see, for example, Means, G. E. et al. (1974)). Among these reagents are, for example, N-succinimidyl 3-(2-pyridyldithio) propionate (SPDP) or N,N′-(1,3-phenylene) bismaleimide.

Cross-linking reagents may be homobifunctional, i.e., having two functional groups that undergo the same reaction such as bismaleimidohexane (“BMH”).

Alternatively, to solve the problems of protein denaturation and contamination during chemical conjugation, recombinant techniques can be used to covalently attach the polypeptide of interest to the virulence factor, such as by joining the nucleic acid coding for the polypeptide of interest with the nucleic acid sequence coding for the virulence factor and introducing the resulting gene construct into a cell capable of expressing the conjugate.

Recombinant methodologies required to produce a DNA encoding a desired protein are well known and routinely practiced in the art. Laboratory manuals, for example MOLECULAR CLONING: A LABORATORY MANUAL. Cold Spring Harbor Press: Cold Spring Harbor, N.Y. (1989) describes in detail techniques necessary to carry out the required DNA manipulations.

The fusion protein can be produced in recombinant microorganism transformed therewith. In this process, each protein component is preferably linked in the molecular ratio of 1:1 (injection domain: catalytic domain). The aid of an appropriate linker, in order to allow proper folding of each protein molecule can be useful. As a linker, it is preferable to use a peptide consisting of the appropriate number of amino acids to maintain activity of each protein component, such as, a peptide composed of 0 to 20 amino acids, though glycine, (glycine)₄ serine, or [(glycine)₄ serine]₂.

Preferable vectors include any of the well known prokaryotic expression vectors, recombinant baculoviruses, COS cell specific vectors, or yeast-specific expression constructs.

Alternatively, the two separate nucleotide sequences can be expressed in a cell or can be synthesized chemically and subsequently joined, using known techniques. Alternatively, the fusion protein can be synthesized chemically as a single amino acid sequence (i.e., one in which both constituents are present) and, thus, joining is not needed.

Preferably, the injection domain of a Rho GTPase activator is a polypeptide selected from the group consisting of:
a polypeptide comprising the amino acid sequence starting at the amino acid residue 1 and ending at the amino acid residue 719 of sequence SEQ ID NO:1;
a polypeptide comprising the amino acid sequence starting at the amino acid residue 1 and ending at the amino acid residue 719 of sequence SEQ ID NO:2;
a polypeptide comprising the amino acid sequence starting at the amino acid residue 1 and ending at the amino acid residue 719 of sequence SEQ ID NO:3; and
a polypeptide comprising the amino acid sequence starting at the amino acid residue 1 and ending at the amino acid residue 1145 of sequence SEQ ID NO:4.

Preferably, the catalytic domain of a Rho GTPase activator is a polypeptide selected from the group consisting of:
a polypeptide comprising the amino acid sequence starting at the amino acid residue 720 and ending at the amino acid residue 1014 of sequence SEQ ID NO:1,
a polypeptide comprising the amino acid sequence starting at the amino acid residue 720 and ending at the amino acid residue 1014 of sequence SEQ ID NO:2,
a polypeptide comprising the amino acid sequence starting at the amino acid residue 720 and ending at the amino acid residue 1014 of sequence SEQ ID NO:3,
a polypeptide comprising the amino acid sequence starting at the amino acid residue 1146 and ending at the amino acid residue 1451 of sequence SEQ ID NO:4,
a polypeptide comprising the amino acid sequence SEQ ID NO:5,
a polypeptide comprising the amino acid sequence SEQ ID NO:6,
a polypeptide comprising the amino acid sequence SEQ ID NO:7,
a polypeptide comprising the amino acid sequence SEQ ID NO:8, and
a polypeptide comprising the amino acid sequence SEQ ID NO:9.

The invention also concerns the vaccine or an immunogenic composition as described above, further comprising one or more antigens against which an immune response is sought.

Indeed, the immunoadjuvant according to the present shall enhance the immune response against the said or more antigens. It goes without saying that the immunoadjuvant is distinct from anyone of said one or more antigens against which the said immune response is sought. Accordingly, in a preferred embodiment, the immunogenic composition or the vaccine composition comprises:
one or more antigens against which an immune response is sought, and
an immunoadjuvant which consists of a Rho GTPase activator able to maintain a Rho GTPase protein in a form bound to GTP;
   wherein the said immunoadjuvant is able to enhance the immune response against the said one or more antigens and wherein the said immunoadjuvant is distinct from anyone of the said one or more antigens.

As illustrated in the following examples, when co-administered with an antigen, an immunoadjuvant as defined in the present invention enhances the humoral response to said antigen.

In some embodiment, the immunoadjuvant is able to enhance the humoral immune response against the one or more antigens comprised in the vaccine or the immunogenic composition.

Preferably, the one or more antigens are selected from the group consisting of a hormone, a protein, a drug, an enzyme, a vaccine composition against bacterial, viral, fungal, prion, or parasitic infections, a component produced by microorganisms, inactivated bacterial toxins such as cholera toxin, ST and LT from Escherichia coli, tetanus toxin from Clostridium tetani, and proteins derived from HIV viruses.

As described above, the one or more antigens are distinct from the Rho GTPase activator used as immunoadjuvant in the vaccine or immunogenic composition.

In some embodiments, the one or more antigens do not comprise the amino acid sequence starting at the amino acid residue 720 and ending at the amino acid residue 1014 of sequence SEQ ID NO:1.

In some embodiments, the one or more antigens do not comprise the amino acid sequence starting at the amino acid residue 720 and ending at the amino acid residue 1014 of sequence SEQ ID NO:2.

In some embodiments, the one or more antigens do not comprise the amino acid sequence starting at the amino acid residue 720 and ending at the amino acid residue 1014 of sequence SEQ ID NO:3.

In some embodiments, the one or more antigens do not comprise the amino acid sequence starting at the amino acid residue 1146 and ending at the amino acid residue 1451 of sequence SEQ ID NO:4.

In some embodiments, the one or more antigens do not comprise the amino acid sequence SEQ ID NO:5.

In some embodiments, none of the one or more antigens comprises comprising the amino acid sequence SEQ ID NO:6.

In some embodiments, the one or more antigens do not comprise the amino acid sequence SEQ ID NO:7.

In some embodiments, the one or more antigens do not comprise the amino acid sequence SEQ ID NO:8.

In some embodiments, none of the one or more antigens comprises comprising the amino acid sequence SEQ ID NO:9.

In some embodiments, the one or more antigens are not antigens from Escherichia coli strains.

In some embodiments, the one or more antigens are not antigens from Bordetella pertussis strains. In some embodiments, the one or more antigens are not antigens from Yersinia pseudotuberculosis strains.

In some embodiments, the one or more antigens are not antigens from Salmonella typhimurium strains.

In some embodiments, the one or more antigens are not antigens from Shigella flexneri strains

In some embodiments, the one or more antigens are not antigens from Helicobacter pylori strains

The amount of antigen, and immunoadjuvant compound in the vaccine composition according to the invention, the dosages administered, are determined by techniques well known to those skilled in the pharmaceutical art, taking into consideration such factors as the particular antigen, the age, sex, weight, species, and condition of the particular animal or patient, and the route of administration.

In a preferred embodiment, the vaccine or immunogenic composition according to the invention, further comprises one or more pharmaceutically acceptable components or excipients, or solid or liquid carriers.

Some of these components or excipients may be selected from the group consisting of surfactants, absorption promoters, water absorbing polymers, substances which inhibit enzymatic degradation, alcohols, organic solvents, oils, pH controlling agents, preservatives, osmotic pressure controlling agents, propellants, water and mixture thereof.

The vaccine or immunogenic composition according to the invention can further comprise a pharmaceutically acceptable carrier. The amount of the carrier will depend upon the amounts selected for the other ingredients, the desired concentration of the antigen, the selection of the administration route, oral or parenteral, etc. The carrier can be added to the vaccine at any convenient time. In the case of a lyophilised vaccine, the carrier can, for example, be added immediately prior to administration. Alternatively, the final product can be manufactured with the carrier.

Examples of appropriate carriers include, but are not limited to, sterile water, saline, buffers, phosphate-buffered saline, buffered sodium chloride, vegetable oils, Minimum Essential Medium (MEM), MEM with HEPES buffer, etc.

Optionally, the vaccine or immunogenic composition of the invention may contain conventional, secondary adjuvants in varying amounts depending on the adjuvant and the desired result. The customary amount ranges from about 0.02% to about 20% by weight, depending upon the other ingredients and desired effect.

Examples of suitable secondary adjuvants include, but are not limited to, stabilizers; emulsifiers; aluminum hydroxide; aluminum phosphate; pH adjusters such as sodium hydroxide, hydrochloric acid, etc.; surfactants such as Tween.®. 80 (polysorbate 80, commercially available from Sigma Chemical Co., St. Louis, Mo.); liposomes; iscom adjuvant; synthetic glycopeptides such as muramyl dipeptides; extenders such as dextran or dextran combinations, for example, with aluminum phosphate; carboxypolymethylene; bacterial cell walls such as mycobacterial cell wall extract; their derivatives such as Corynebacterium parvum; Propionibacterium acne; Mycobacterium bovis, for example, Bovine Calmette Guerin (BCG); vaccinia or animal poxvirus proteins; subviral particle adjuvants such as orbivirus; cholera toxin; N,N-dioctadecyl-N′,N′-bis(2-hydroxyethyl)-propanediamine (pyridine); monophosphoryl lipid A; dimethyldioctadecylammonium bromide (DDA, commercially available from Kodak, Rochester, N.Y.); synthetics and mixtures thereof. Desirably, aluminum hydroxide is admixed with other secondary adjuvants or an immunoadjuvant such as Quil A.

Examples of suitable stabilizers include, but are not limited to, sucrose, gelatin, peptone, digested protein extracts such as NZ-Amine or NZ-Amine AS. Examples of emulsifiers include, but are not limited to, mineral oil, vegetable oil, peanut oil and other standard, metabolizable, nontoxic oils useful for injectables or intranasal vaccines compositions.

For the purpose of this invention, these adjuvants are identified herein as “secondary” merely to contrast with the above-described immunoadjuvant compound, consisting of a Rho GTPase activator, that is an essential ingredient in the vaccine composition for its effect in combination with an antigenic substance to significantly increase the humoral immune response to the antigenic substance. The secondary adjuvants are primarily included in the vaccine formulation as processing aids although certain adjuvants do possess immunologically enhancing properties to some extent and have a dual purpose.

Conventional preservatives can be added to the vaccine composition in effective amounts ranging from about 0.0001% to about 0.1% by weight. Depending on the preservative employed in the formulation, amounts below or above this range may be useful. Typical preservatives include, for example, potassium sorbate, sodium metabisulfite, phenol, methyl paraben, propyl paraben, thimerosal, etc.

The choice of inactivated, modified or other type of vaccine composition and method of preparation of the improved vaccine composition formulation of the present invention are known or readily determined by those of ordinary skill in the art.

A pharmacologically effective amount of the immunoadjuvant compound according to the invention may be given, for example orally, parenterally or otherwise, concurrently with, sequentially to or shortly after the administration of a an antigenic substance in order to potentiate, accelerate or extend the immunogenicity of the antigen.

While the dosage of the vaccine or immunogenic composition depends upon the antigen, species, body weight of the host vaccinated or to be vaccinated, etc., the dosage of a pharmacologically effective amount of the vaccine composition will usually range from about 50 μg to about 500 μg per dose, per kilogram of body weight, in a mouse model.

Although the amount of the particular antigenic substance in the combination will influence the amount of the immunoadjuvant compound according to the invention, necessary to improve the immune response, it is contemplated that the practitioner can easily adjust the effective dosage amount of the immunoadjuvant compound through routine tests to meet the particular circumstances.

As a general rule, the vaccine or the immunogenic composition of the present invention is conveniently administered orally, parenterally (subcutaneously, intramuscularly, intravenously, intradermally or intraperitoneally), intrabuccally, intranasally, or transdermally. The route of administration contemplated by the present invention will depend upon the antigenic substance and the co-formulants. For instance, if the vaccine composition contains saponins, while non-toxic orally or intranasally, care must be taken not to inject the sapogenin glycosides into the blood stream as they function as strong hemolytics. Also, many antigens will not be effective if taken orally. Preferably, the vaccine composition is administered subcutaneously, intramuscularly or intranasally.

The dosage of the vaccine or the immunogenic composition will be dependent upon the selected antigen, the route of administration, species, body weight and other standard factors. It is contemplated that a person of ordinary skill in the art can easily and readily titrate the appropriate dosage for an immunogenic response for each antigen to achieve the effective immunizing amount and method of administration.

The inventors have also shown, in example 1 that CNF1 has Immunoadjuvant properties when coadministered orally with an antigen. They have also shown that this coadministration enhances the total IgA antibody titer in mice. This last result is typical of a mucosal response to an immunisation.

Consequently, a further object of the invention is a vaccine or an immunogenic composition according to the invention, for administration to a mucosal surface.

This mode of administration presents a great interest. Indeed, the mucosal membranes contain numerous of dendritic cells and Langerhans cells, which are excellent antigen detecting and antigen presenting cells. The mucosal membranes are also connected to lymphoid organs called mucosal associated lymphoid tissue, which are able to forward an immune response to other mucosal areas. An example of such an epithelia is the nasal epithelial membrane, which consists of practically a single layer of epithelial cells (pseudostratified epithelium) and the mucosal membrane in the upper respiratory tract is connected to the two lymphoid tissues, the adenoids and the tonsils. The extensive network of blood capillaries under the nasal mucosal of the high density of B and T cells, are particularly suited to provide a rapid recognition of the antigen and provide a quick immunological response.

Preferably, the mucosal surface is selected from the group consisting of mucosal surfaces of the nose, lungs, mouth, eye, ear, gastrointestinal tract, genital tract, vagina, rectum, and the skin.

Another object of the invention is a vaccine or an immunogenic composition for an oral administration.

The invention concerns also a protein comprising a polypeptide consisting of; from the N-terminal end to the C-terminal end, respectively:
a) the injection domain of a Rho GTPase activator as described above, and
b) the catalytic domain of a Rho GTPase activator as described above.

The invention further concerns the use of a polypeptide of interest, for manufacturing a vaccine or an immunogenic composition.

The invention also concerns the use of a fusion protein as described above for manufacturing a vaccine or an immunogenic composition.

Another object of the invention is to provide a method for preparing an immunogenic or a vaccine composition as defined above, the said method comprising the steps of:
(i) providing one or more antigens against which an immune response is sought
(ii) providing an immunoadjuvant as defined in anyone of claims 1 to 9 and
(iii) mixing the said one or more antigens from step (i) and the immunoadjuvant from step (ii) optionally in the presence of one or more pharmaceutically acceptable excipients.

As described herein, the said immunoadjuvant is able to enhance the immune response against the said one or more antigens and the said immunoadjuvant is distinct from anyone of the said one or more antigens.

The invention further relates to a method for inducing an immune response to a patient in need thereof, the said method comprises the step of:
(i) providing an immunogenic composition as described above and
(ii) administering the immunogenic composition provided in step (i) to a patient in need thereof.

In some embodiments, the method for inducing an immune response comprises the administration of the said immunogenic composition by the mucosal route.

In other embodiments, the method for inducing an immune response comprises the administration of the immunogenic composition by the oral route.

In some embodiments, the method for inducing an immune response is a method for immunizing a patient in need and thus the immunogenic composition is a vaccine composition.

Further details of the invention are illustrated in the following non-limiting examples.

### Materials and Methods

### Cells and Reagents

Human umbilical vein endothelial cells (HUVEC) were obtained from PromoCell (Heidelberg, Germany). Cells were grown in Human Endothelial SFM medium (Invitrogen Co, Paisley, Scotland) supplemented with defined growth factors (d-SFM): 10 ng/ml EGF and 20 ng/ml bFGF (Invitrogen Co), 1 μg/ml heparin (Sigma-Aldrich) and either 20% fetal bovine serum (Invitrogen Co) or 1% (W/V) bovine serum albumin (ELISA grade, Sigma-Aldrich) together with penicillin and streptomycin (Invitrogen Co). Cells were grown on 0.2% gelatine coated dishes (Sigma-Aldrich). Transfections of HUVEC were carried out as described by Mettouchi et al., 2001. Antibodies used were monoclonal anti-β actin antibody [clone AC-74] (Sigma-Aldrich); anti-RhoA, anti-Cdc42, anti-Rac1 and anti-Ras antibodies (Transduction Laboratories); anti-HA [clone 11] (BabCO); anti-E-selectin [clone CTB202] (Santa Cruz Biotechnology) and rabbit polyclonal anti-phospho-p44/42 MAP kinase (Thr202/Tyr204), anti phospsho-p38 MAP kinase (Thr180/Tyr182) and anti phospho-c-Jun (Ser73) (Cell Signaling Technology); anti-human IκB-α (Upstate Biotechnology); anti-TRAF1 (H-186, Santa Cruz Biotechnology). Primary antibodies were visualized using goat anti-mouse or anti-rabbit horseradish peroxidase-conjugated secondary antibodies (DAKO, Glostrup, Denmark). TRAF1 rabbit antibodies were visualized using biotin-XX goat anti-rabbit IgG followed by streptavidin horseradish peroxidase conjugate (Molecular Probes). DNA vectors corresponding to pcDNA3RhoQ63L, RacQ61 L and Cdc42061L were provided by Manor, D. (Lin et al., 1999).

### Toxins

Purified CT was obtained from List Biologicals (Campbell, Calif.). CNF1 and CNF1-C866S toxins production and purification were performed as previously described (Munro et al., 2004). Briefly, overnight cultures of E. coli OneShot, carrying pCR2cnf1 or pCR2cnf1C866S were lysed in PBS using a French Press. After ammonium sulfate precipitation and dialysis against Tris-NaCl buffer, the soluble fraction was then applied to series of column purifications. Protein purification was followed by SDS-PAGE. The activity of the different batches of CNF1 toxin was estimated by multinucleation assay, as previously described Lemichez et al., 1997). The purified CNF1 toxin used in this study produced, at 10-12 M, 50% of multinucleation of HEp-2 cells after 48 h of exposure. CNF1 catalytic domain (amino acids 720-1014) and DNT catalytic domain (amino acids 1154-1451) were produced using the same methods and activities were assessed as described earlier for CNF1. All protein preparations were found to contain doses of endotoxin below 0.12 EU/ml of FDA Reference Standard, using the Multi-Test Limulus Amebocyte Lysate Pyrogen Plus® (Biowhittaker, Walkersville, Md.). Activation and degradation of Rac was assessed using GST-protein pulldown experiment as previously described (Doye et al., 2002).

### Immunizations

FemalesBALB/c mice were purchased from Charles River Laboratories (L'Arbresle, France). They were maintained and handled according to the regulations of the European Union and the French Department of Health. In all experiments, 4-8 week-old female mice were used. Mice were fed either CNF1, CNF1-C866S (a catalytic inactive toxin), catalytic domains of CNF1 (CNF-CD) and DNT (DNT-CD) or CT in the presence or absence of 5 mg of ovalbumin (OVA) (grade V, Sigma-Aldrich, St. Louis, Mo.) dissolved in a solution of 500 μl of 3% NaHCO₃. Animals were fed on either two or three consecutive occasions, as detailed in figure legends, 10-12 days apart.

### Measurements of Serum Antibody Responses

Serum antibody levels against OVA were determined by means of solid-phase ELISA, as previously described (anjuére et al., 2003). Briefly, serial three-foldilutions of test and control sera were incubated for 2 h at room temperature in OVA-coated polystyrene microtiter wells (Nunc-Immuno™ Plates, MaxiSorp™ Surface, Nunc, Denmark). After washes with PBS containing 0.05% Tween, wells were exposed to 0.1 ml of PBS-Tween containing appropriately diluted HRP-conjugated goat anti-mouse IgG, IgG1, IgG2a, IgG2b and IgA (Southern Biotech Inc., Birmingham, Ala.). Plates were developed with BM blue, POD chromogenic substrate (Roche Applied Science, Indianapolis, Ind.) and monitored spectrophotometrically. Titers were defined as the reciprocal of the highest dilution of serum giving an absorbance value of twice above control, corresponding to pre-immune serum.

### Measurements Of Mucosal Antibody Responses

Six days after the last immunization, mice were anesthetized with entobarbital and injected with pyrogen-free isotonic saline containing 100 units heparin. The carotid vein was cut and animals were perfused in situ with 25 ml of PBS containing 100 units/ml heparin administered by intracardiac injection to minimize contamination with blood. The small intestines were resectioned, opened longitudinally and washed with PBS. Sections were cut into small fragments and further perfused with PBS-heparin for 4 h at 4° C. Tissue fragments were weighed and then manipulated according to the PERFEXT method, based upon sequential perfusion and detergent extraction (Villavedra et al., 1997). Briefly, fragments were homogenized, suspended in 2 ml of extraction buffer/mg of tissue and incubated overnight at 4° C. The extraction buffer consisted of PBS supplemented with 2% saponin (Sigma) and protease inhibitors (Complete, Boehringer). Samples were then kept frozen at −80° C. until assayed. Thirty minutes before use, specimens were allowed to thaw at room temperature and spun at 16,000×g for 10 min. Supernatants were assayed for IgA and IgG anti-OVA antibody titers as described earlier.

### Histology

Mice were fed CNF1 or CNF1-C866S. After 48 h, mice were killed and the small intestines were collected, fixed in formalin and embedded in paraffin wax. Consecutive 5 μm paraffin sections were stained with haematoxylin and eosin.

### DNA Array Analysis

HUVEC were seeded at 8 10⁶ cells/150 mm gelatin-coated dish in d-SFM containing BSA. Cells were intoxicated in parallel for 3 h and 24 h in d-SFM/BSA supplemented with 10⁻⁹M CNF1. Cells were lysed in RTL buffer for total RNA extraction, according to the manufacturer (RNeasy MiniKit, Qiagen). CNF1 regulated genes were analyzed using Affymetrix® Human GeneChip U133A and U133B, by Aros Applied Biotechnology ApS (www.arosab.com), as recommended by the manufacturer (www.Affymetrix.com).

### ELISA

HUVEC were seeded 24 h before toxin addition at 2 10⁵ cells/22.5 mm or 5 10⁵ cells/35 mm well in d-SFM containing serum. Intoxication of cells was performed by addition of fresh medium containing CNF1, for different periods of time. One hour before intoxication ending the medium was replaced by d-SFM containing BSA for ELISA. IL-8, MCP-1, IL-6, MIP3-α, TNF-α and RANTES production were assessed using human Quantikine® immunoassays, as recommended by the manufacturer (R & D Systems, Abingdon, UK).

### Pull-Down and Immunoblotting Detection of Activated-Rho GTPases

Levels of activated-RhoA, -RhoB, -RhoC, -Rac1, -Rac2, -Cdc42 were measured using classical Rho effector pull-down assays developed by Manser et al., 1998 and Ren et al., 1999. For antibodies description see the cells and reagents section.
Briefly, the measure of the levels of activated-RhoA, -B and -C was performed as followed. Cells were lysed in 50 mM Tris, pH7.2, 500 mM NaCl, 10 mM MgCl2, 1% Triton X-100, 0.5% deoxicholate, 0.1% SDS and protease inhibitors. Cell lysates were clarified by centrifugation at 13000 g at 4° C. for 10 min. and equal volumes of lysates (corresponding to 1 mg of total proteins) were incubated with 30 micrograms GST-RBD (Rho binding domain of Rhotekin fused to GST and described in Ren et al., 1999) beads at 4° C. for 45 min. The beads were washed four times with buffer B (50 mM Tris, pH7.2, 500 mM NaCl, 10 mM MgCl2, 1% Triton X-100 and protease inhibitors). Bound Rho proteins were resolved by SDS-PAGE and transferred on PVDF membranes. Activated-Rho proteins were detected by immunoblotting using a monoclonal antibody against either RhoA and RhoC or RhoB and anti-mouse horseradish peroxidase-conjugated secondary antibody followed by chemiluminescence detection.
The measure of the levels of activated-Rac1, Rac2 and Cdc42 was determined, as followed. Cells were lysed in LB buffer (25 mM Tris, pH7.5, 150 mM NaCl, 5 mM MgCl2, 0.5% Triton X-100, 4% glycerol and protease inhibitors). Cell lysates were clarified by centrifugation at 13000 g at 4° C. for 10 min. and equal volumes of lysates (corresponding to 1 mg of total proteins) were incubated with 30 micrograms GST-PAK70-106 (Rac/Cdc42 binding domain of p21 PAK fused to GST and described in Manser et al., 1998) beads at 4° C. for 45 min. The beads were washed four times with LB. Bound Rac and Cdc42 proteins were resolved by SDS-PAGE and transferred on PVDF membranes. Activated-Rac1, 2 or activated-Cdc42 proteins were detected by immunoblotting using a monoclonal antibody against either Rac1, 2 or Cdc42 and anti-mouse horseradish peroxidase-conjugated secondary antibody followed by chemiluminescence detection.
For activated Ras measurements GST-RBD1-149 of Raf1 was used as described by the authors (de Rooij and Bos, 1997).

### Expression of Activated or Inactivated Rho GTPase by THP1 Cells and Measurement of the Resulting IL1-β Secretion

THP1 cells were transfected using the Nucleofector II (Amaxa, Cologne, Germany) and the Nucleofector Kit V (Amaxa) according to manufacturer's instructions. The number of cells was adjusted to 10⁶ and then transfected with 1 μg of each DNA construct i.e. pRK5-Myc-RhoA V14 or N19, pRK5-Myc-Rac1 L61 or N17, pRK5-Myc-Rac2 L61 or N17, pRK5-Myc-Cdc42 L61 or N17 and pEGFP. Control cells correspond to THP1 cells transfected with the DNA construct encoding for GFP.

RhoA V14, Rac1 L61, Rac2 L61 and Cdc42 L61 are constitutively activated Rho GTPase mutants, whereas RhoA N19, Rac1 N17, Rac2 N17 and Cdc42 N17 are inactive Rho GTPase mutants.

At 16 hours post-transfection, cells were lysed with 9 mM CHAPS, and IL-1β production was assayed by ELISA. The assay used anti-IL-1β sheep IgG coated onto a microtiter plate, as the capture antibody, and HRP-labelled sheep Fab′ anti-IL-1β, as the second antibody, (method described in Ferrua, B., P. Becker, L. Schaffar, A. Shaw, and M. Fehlman. 1988. Detection of human IL-1α and IL-1β at the subpicomolar level by colorimetric sandwich enzyme immunoassay. J. Immunol. Methods 114:41-48).

The threshold sensitivity was of 20 pg IL-1β/ml and the assay recognized equally well the 31 kDa IL-1β precursor and the 17 kDa mature secreted forms, as previously described in Ferrua et al, (1988).

### Example 1

### CNF1 Effects on Cell Signaling Pathways

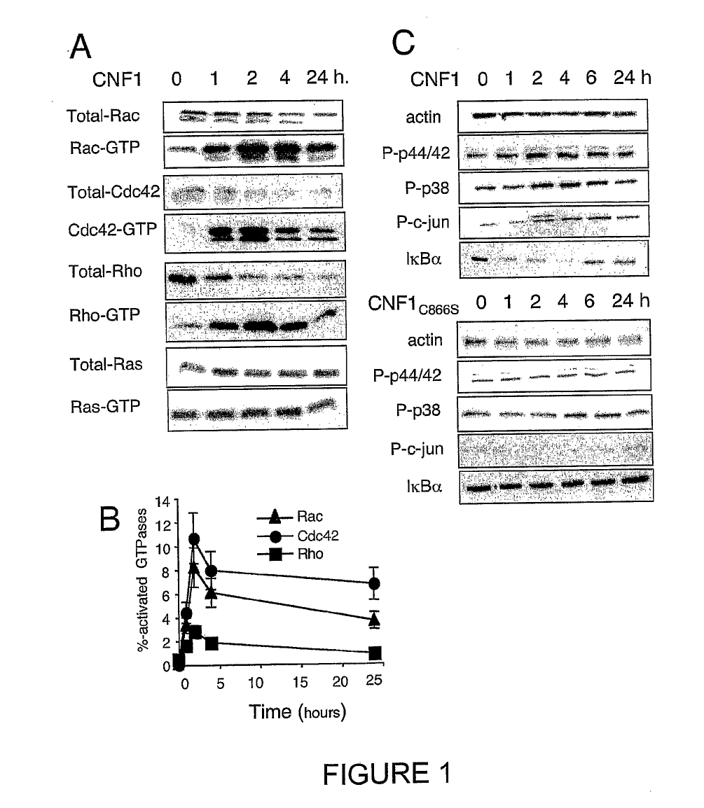

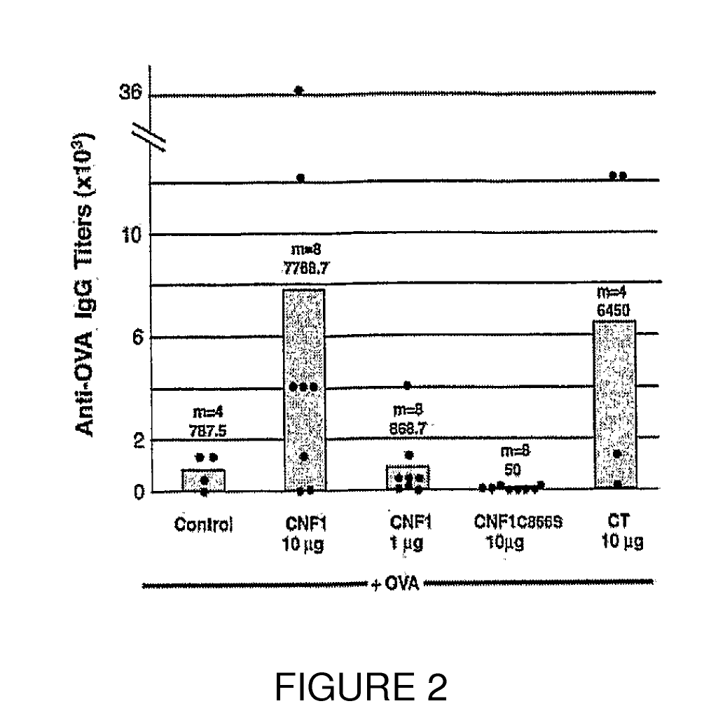

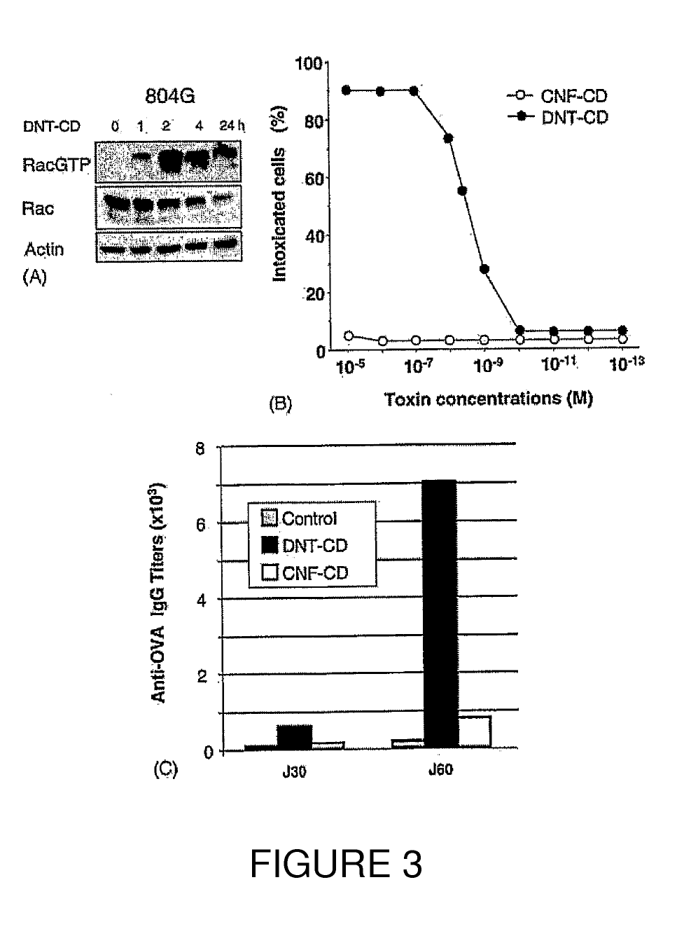

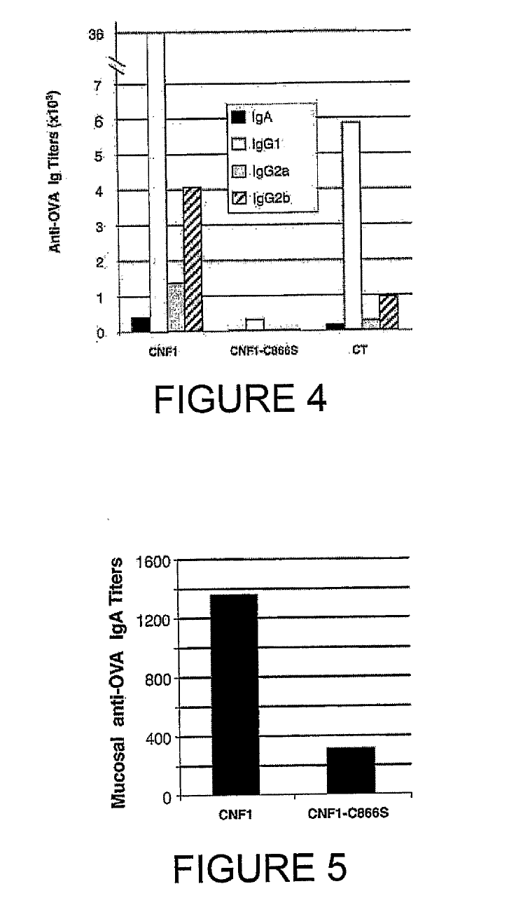

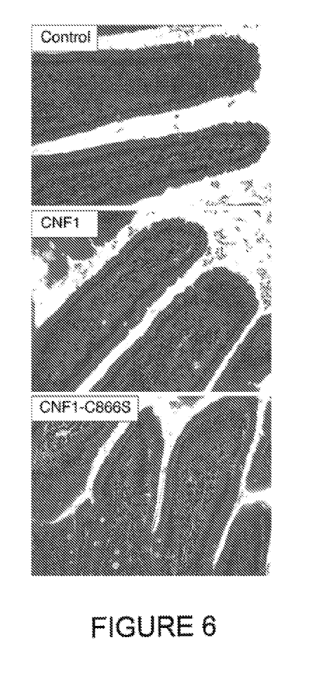

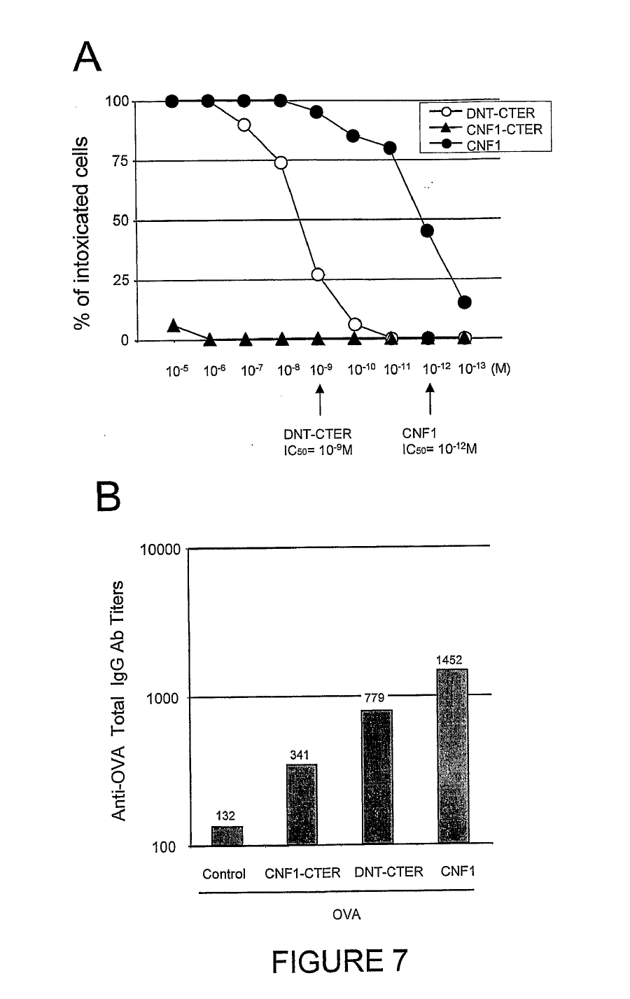

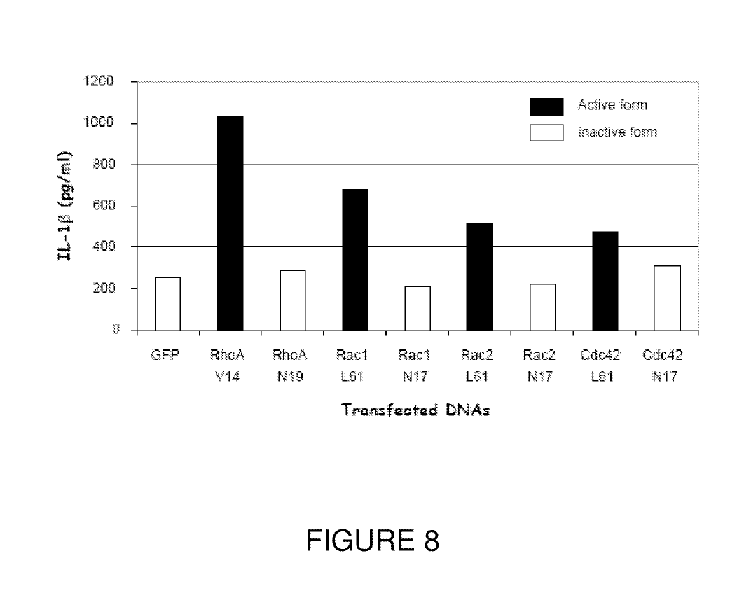

Kinetics of CNF1-induced Rac1, Cdc42 and RhoA activation have been studied. These kinetics show the specificity of Rho protein activation, as compared to the Ras GTPase (FIG. 1A, 1B). Obviously, these measurements do not represent an exhaustive list of the Rho proteins activated by CNF1, other Rho bearing the canonical sequence for CNF1 recognition/modification (Lerm et al., 1999). These measurements rather indicated that all the three Rho proteins exhibited a maximal activation around 2 hours in HUVEC intoxicated with 10⁻⁹M CNF1 (FIG. 1B). CNF1 interference with classical signaling pathways leading to gene regulation, has also been shown. Consistent with the absence of Ras activation measured, CNF1 did not produce ERK1/2 phosphorylation (FIG. 1A, 1C). CNF1 rather appeared to interfere both with the SAP-kinase signaling pathways, unraveled by p38MAP-kinase and cjun phosphorylations. CNF1 also interferes with the NF-kappaB pathway, as shown by IkB depletion (FIG. 1C). Host cells have evolved cell surface receptors to get alarmed of the presence of PAMP (Medzhitov and Janeway, 2002). PAMP receptors initiate an innate immune response through IkB depletion for NFkB activation (Barton and Medzhitov, 2003). That cell treatment with the catalytic inactive CNF1-C866S toxin was devoid of interference with all signaling pathways tested, especially NFkB, strongly suggested an absence of cell recognition of CNF1 as a PAMP (FIG. 1C).

### Example 2

### Serum Anti-OVA Response Following Mucosal Immunization of Mice Co-Fed CNF1

Using a mouse model, characteristics of the host humoral response to CNF1 were investigated. Animals orally immunized with OVA, a prototype soluble antigen, co-administered with CNF1 (10 μg) displayed serum IgG anti-OVA antibody responses (geometric mean titer 7768.7) comparable to those elicited by cholera toxin (geometric mean titer 6450) (FIG. 2). Under these experimental conditions, no serum anti-CNF1 responses were detected (not shown). It was also verified that neither CNF1 nor CT alone elicited production of seric anti-OVA IgG antibodies (not shown). Immunization with a lower dose of CNF1 (1 μg) had negligible effects on serum anti-OVA responses when compared to control animals (geometricmean titers 868.7 and 787.5, respectively) (FIG. 2). Finally, immunization with 10 μg of the catalytic inactive CNF1 mutant (CNF1-C866S) failed to enhance serum anti-OVA responses in animals co-fed OVA (FIG. 2). This result together with the fact that both CNF1 and CNF1-C866S were purified using identical conditions, excludes a possible stimulation of the IgG anti-OVA antibody responses by factors co-purified with CNF1. Collectively, these results show that the anti-OVA response elicited by CNF1 is dose dependent and requires its catalytic activity. As described for CNF1 (Doye et al., 2002), the catalytic domain of the closely related toxin DNT (DNT-CD) produced a transient activation of Rac due to the cellular depletion of this GTPase (FIG. 3A). Effects of DNT-CD were quantified using a classical HEp-2 cell assay, which gives a 50% multinucleation of cells at 10-12M CNF1 (Lemichez et al., 1997). In contrast to DNT-CD, which showed a 50% effect at 10−9 M, the CNF1 catalytic domain CNF-CD had negligible effects (FIG. 3B). When immunostimulatory effects of both catalytic domains CNF-CD and DNT-CD are compared, only mice immunized with 100 μg of DNT-CD developed a significant level of serum IgG anti-OVA antibodies (DNT-CD geometric mean titer of 7015 at 60 days) (FIG. 3C).

### Example 3

### Serum Antibody Isotype Responses

Sera from mice immunized with OVA together with 10 μg of CNF1, CNF1-C866S or CT were then tested for the presence of anti-OVA IgA and IgG subclasses. The isotype distribution of 1 g anti-OVA antibody responses in animals immunized with CNF1 was similar to that observed in animals immunized with CT and was mainly accounted for by IgG1 and IgG2b. Likewise, mice fed a mixture of OVA and CNF1-C866S had no detectable anti-OVA antibody responses in any isotype (FIG. 4). Taken together, these results indicate that CNF1, when given orally with OVA, promotes systemic anti-OVA responses with a profile of IgG subclasses similar to that induced by cholera toxin.

### Example 4

### Mucosal IgA Antibody Response

Using the PERFEXT method, we then evaluated the ability of CNF1 to potentiate mucosal antibody responses in animals orally immunized with OVA. Sections of small intestine collected from groups of mice orally immunized with OVA, given together with CNF1 or CNF1-C866S, were analyzed for IgA content 2 weeks after the last of three immunizations. As illustrated in FIG. 5, oral co-administration of OVA with CNF1 elicited an antigen specific mucosal IgA response. Mice orally immunized with OVA given alone or admixed with the catalytic inactive CNF1-C866S had no detectable intestinal IgA antibody responses to OVA (FIG. 5).

### Example 5

### Histological Analysis of CNF1 Effects on Small Intestines

Histological analyses of sections of small intestines prepared from mice immunized with CNF1 or CNF1-C866S showed no significant differences to those from control (bicarbonate fed) animals (FIG. 6).

### Example 6

### The Catalytic Domain of DNT Remains Active on Cells and is Sufficient to Confer Adjuvanticity

CNF1 belongs to a family of toxins among them DNT, having similar catalytic activity (Boquet and Lemichez 2003). It is shown on FIG. 3A that the catalytic domain of DNT (DNT-CTER) remains active on cells, although showing a lower intoxication property as compared to CNF1. Despite its inability to intoxicate cells (FIG. 7A), the catalytic domain of CNF1 (CNF1-CTER) upon mechanical injection into cells produces a bona fide toxic phenotype (Lemichez et al., 1997). It has been taken advantage of the above observations to test the adjuvant properties of the catalytic domains of both toxins. Mice were fed 10 times higher quantities of both toxin catalytic domains, as compared to CNF1. In these conditions it has been observed that DNT-CTER stimulated significantly the anti-OVA IgG responses (FIG. 7B). CNF1-CTER also produced a stimulation of the anti-OVA IgG responses, although at a lower level (FIG. 7B). Taken together, these results indicate that the adjuvanticity of this group of toxin is encompassed in their catalytic domain. Nevertheless, the injection domain of CNF1-toxin together with its catalytic domain, allows the use of lower doses to induce a significantly higher biological effect.

### Example 7

### Expression of Constitutively Active Forms of RhoA, Rac1, Rac2 or Cdc42 in THP-1 Cells Induces the Production of IL1-β

As illustrated in FIG. 8, the expression of activated forms of RhoA, Rac1, Rac2 or Cdc42 in THP1 cells, induce a significant increase of secretion of IL-1β as compared to control cells. On the contrary, the secretion of IL-1β for THP1 cells expressing an inactive Rho GTPase is similar to that of control cells.

The experimental results of Example 7 clearly show that the activation of Rho GTPase induces the secretion of IL-β which is a pro-inflammatory chemokine with immunoadjuvant property (Staats, 1999).

These results, taken together with the fact that CNF1-C866S (which does not activate Rho GTPases) does not exhibit any immunoadjuvant property, clearly support that the immunoadjuvant property of CNF1 and DNT results from their functional feature their ability to activate Rho-GTPase.

### REFERENCES

Anjuére, F., George-Chandy, A., Audant, F., Rousseau, D., Holmgren J., and Czerkinsky, C. (2003). Transcutaneous immunization with cholera toxin B subunit adjuvant suppresses IgE antibody responses via selective induction of Th1 immune responses. J. Immunol. 170, 1586-1592.
Arbibe, L., Mira, J. P., Teusch, N., Kline, L., Guha, M., Mackman, N., Godowski, P. J., Ulevitch, R. J., and Knaus, U. G. (2000). Toll-like receptor 2-mediated NF-kappa B activation requires a Rac1-dependent pathway. Nat. Immunol. 1, 533-540.
Baggiolini, M., and Loetscher, P. (2000). Chemokines in inflammation and immunity. Immunol. Today 21, 418-420.
Barbieri J. T., Riese M. J., Aktories, K. (2002). Bacterial toxins that modify the actin cytoskeleton. Annu. Rev. Cell. Dev. Biol. 18, 315-344.
Barnhart, B. C., and Peter, M. E. (2003). The TNF receptor 1: a split personality complex. Cell 114, 148-150.
Barton, G. M. and Medzhitov, R. (2003). Toll-like receptor signaling pathways. Science 300, 1524-1525.
Bishop, A. L., and Hall, A. (2000). Rho GTPases and their effector proteins. Biochem. J. 348, 241-255.
Boquet, P., and Lemichez, E. (2003). Bacterial virulence factors targeting Rho GTPases: parasitism or symbiosis? Trends Cell Biol. 13, 238-246.
Burridge, K., and Wennerberg, K. (2004). Rho and Rac take center stage. Cell 116, 167-179
Chimini, G., and Chavrier, P. (2000). Function of Rho family proteins in actin dynamics during phagocytosis and engulfment. Nat. Cell Biol. 2, 191-196.
Dieu, M. C., Vanbervliet, B., Vicari, A., Bridon, J. M., Oldham, E., Ait-Yahia, S., Briere, F., Zlotnik, A., Lebecque, S., and Caux, C. (1998). Selective recruitment of immature and mature dendritic cells by distinct chemokines expressed in different anatomic sites. J. Exp. Med. 188, 373-386.
Doye, A., Mettouchi, A., Bossis, G., Clement, R., Buisson-Touati, C., Flatau, G., Gagnoux, L., Piechaczyk, M., Boquet, P., and Lemichez, E. (2002). CNF1 exploits the ubiquitinproteasome machinery to restrict Rho GTPase activation for bacterial host cell invasion. Cell 111, 553-564.
De Rooij, J., and Bos, J. L. (1997). Minimal Ras-binding domain of Raf1 can be used as an activation-specific probe for Ras. Oncogene 14, 623-625.
Etienne-Manneville, S., and Hall, A. (2002). Rho GTPases in cell biology. Nature 420, 629-635.
Ferrua, B., P. Becker, L. Schaffar, A. Shaw, and M. Fehlman. 1988. Detection of human IL-1α and IL-1β at the subpicomolar level by colorimetric sandwich enzyme immunoassay. J. Immunol. Methods 114:41-48
Flatau, G., Lemichez, E., Gauthier, M., Chardin, P., Paris, S., Fiorentini, C., and Boquet, P. (1997). Toxin-induced activation of the G protein p21 Rho by deamidation of glutamine. Nature 387, 729-733.
Galan, J. E., and Zhou, D. (2000). Striking a balance: modulation of the actin cytoskeleton by Salmonella. Proc. Natl. Acad. Sci. USA 97, 8754-8761.
Garrett, W. S., Chen, L. M., Kroschewski, R., Ebersold, M., Turley, S., Trombetta, S., Galan, J. E. and Mellman, I. (2000). Developmental control of endocytosis in dendritic cells by Cdc42. Cell 102, 325-334.
Holmgren, J., Czerkinsky, C., Eriksson, K. and Mharandi, A. (2003). Mucosal immunisation and adjuvants: a brief overview of recent advances and challenges. Vaccine 21, S89-95.
Izadpanah, A., Dwinell, M. B., Eckmann, L., Varki, N. M., and Kagnoff, M. F. (2001). Regulated MIP-3alpha/CCL20 production by human intestinal epithelium: mechanism for modulating mucosal immunity. Am. J. Physiol. Gastrointest. Liver Physiol. 280, 710-719.
Janeway, C. A. Jr. (2001). How the immune system works to protect the host from infection: a personal view. Proc. Natl. Acad. Sci. USA. 98, 7461-7468.
Khan, N. A., Wang, Y., Kim, K. J., Chung, J. W., Wass, C. A., and Kim, K. S. (2002). Cytotoxic necrotizing factor-1 contributes to Escherichia coli K1 invasion of the central nervous system J. Biol. Chem. 277, 15607-15612.
Klein, S., de Fougerolles, A. R., Blaikie, P., Khan, L., Pepe, A., Green, C. D., Koteliansky, V., and Giancotti, F. G. (2002). Alpha 5 beta 1 integrin activates an NF-kappa B-dependent program of gene expression important for angiogenesis and inflammation. Mol. Cell. Biol. 22, 5912-5922.
Krawczyk, C., and Penninger, J. M. (2001). Molecular controls of antigen receptor clustering and autoimmunity. Trends Cell Biol. 11, 212-220.
Kubori, T, and Galan, J. E. (2003). Temporal regulation of salmonella virulence effector function by proteasome-dependent protein degradation. Cell 115, 333-342.
Lemichez, E., Flatau, G., Bruzzone, M., Boquet, P., and Gauthier, M. (1997). Molecular localization of the Escherichia coli cytotoxic necrotizing factor CNF1 cell-binding and catalytic domains. Mol. Microbiol. 24, 1061-1070.
Lerm, M., Schmidt, G., Goehring, U. M., Schirmer, J., and Aktories, K. (1999). Identification of the region of rho involved in substrate recognition by Escherichia coli cytotoxic necrotizing factor 1 (CNF1). J. Biol. Chem. 274, 28999-9004.
Lin, R., Cerione, R. A., and Manor, D. (1999). Specific contributions of the small GTPases Rho, Rac, and Cdc42 to Dbl transformation. J. Biol. Chem. 274, 23633-23641.
Manser, E., Loo, T. H., Koh, C. G., et al., (1998) PAK kinases are directly coupled to the PIX family of nucleotide exchange factors. Mol. Cell. vol 1 pp 183-192.
Means, G. E. and Feeney, R. E., (1974) Chemical Modification of Proteins, Holden-Day, pp. 39-43
Medzhitov, R., and Janeway, C. A. (2002). Decoding the patterns of self and nonself by the innate immune system. Science 296, 298-300.
Mettouchi, A., Klein, S., Guo, W., Lopez-Lago, M., Lemichez, E., Westwick, J. K., and Giancotti, F. G. (2001). Integrin-specific activation of Rac controls progression through the G(1) phase of the cell cycle. Mol. Cell. 8, 115-127.
Müller A., Hoffmann C., Galle M., Van Den Broeke A., Heikenwalder M., Falter L., Misselwitz B., Kremer M., Beyaert R., and Hardt W. D., (2009), The S. typhimurium Effector SopE Induces Caspase-1 Activation in Stromal Cells to Initiate Out Inflammation, Cell Host & Microbe, 6, 125-136
Munro P, Flatau G, Doye A, Boyer L, Oregioni O, Mege J L, et al. Activation and proteasomal degradation of Rho GTPases by CNF1 elicit a controlled inflammatory response. J Biol Chem 2004; 279:35849-57.
Mysorekar, I. U., Mulvey, M. A., Hultgren, S. J., and Gordon, J. I. (2002). Molecular regulation of urothelial renewal and host defenses during infection with uropathogenic Escherichia coli. J. Biol. Chem. 277, 7412-7419.
Olson, M. F., Pasteris, N. G., Gorski, J. L. and Hall A. (1996). Faciogenital dysplasia protein (FDG1) and Vav, two related proteins required for normal embryonic development, are upstream regulators of Rho GTPases. Curr Biol. 6, 1628-1633.
Pedron, T., Thibault, C., and Sansonetti, P. J. (2003). The invasive phenotype of Shigella flexneri directs a distinct gene expression pattern in the human intestinal epithelial cell line Caco-2. J. Biol. Chem. 278, 33878-33886.
Powrie F., and Maloy, K. J. (2003). Immunology. Regulating the regulators. Science 299, 1030-1031.
Ren X. D., Kiosses W. B., and Schwartz M. A., (1999) Regulation of the small GTP-binding protein Rho by cell adhesion and the cytoskeleton. EMBO J. vol 18 pp 578-595
Shapiro, S. D. (2003). Immunology: Mobilizing the army. Nature 421, 223-224.
Schmidt, G., Sehr, P., Wilm, M., Selzer, J., Mann, M., and Aktories, K. (1997). Gln 63 of Rho is deamidated by Escherichia coli cytotoxic necrotizing factor-1. Nature 387, 725-729.
Schmidt, A., and Hall, A. (2002). Guanine nucleotide exchange factors for Rho GTPases; turning on the switch. Genes Dev. 16, 1587-1609.
Staats H. F. and Ennis F. A., IL-1 Is an Effective Adjuvant for Mucosal and Systemic Immune Responses When Coadministered with Protein Immunogens (1999), Journal of Immunology, 162, 6141-6147.
Szyperski, T., Fernandez, C., Mumenthaler, C., and Wuthrich, K. (1998). Structure comparison of human glioma pathogenesis-related protein GliPR and the plant pathogenesis related protein P14a indicates a functional link between the human immune system and a plant defense system. Proc. Natl. Acad. Sci. USA 95, 2262-2266.
Takai, Y., Sasaki, T., and Matozaki, T. (2001). Small GTP-binding proteins. Physiol. Rev. 81, 153-208.
Tran Van Nhieu, G., Bourdet-Sicard, R., Dumenil, G., Blocker, A., and Sansonetti, P. J. (2000). Bacterial signals and cell responses during Shigella entry into epithelial cells. Cell Microbiol. 2, 187-193.
Villavedra, M., Carol, H., Hjulstrom, M., Holmgren, J., and Czerkinsky, C. (1997). “PERFEXT”: a direct method for quantitative assessment of cytokine production in vivo at the local level. Res. Immunol. 148, 257-266.
Walmsley, M. J., Ooi, S. K., Reynolds, L. F., Smith, S. H., Ruf, S., Mathiot, A., Vanes, L., Williams, D. A., Cancro, M. P., and Tybulewicz, V. L. (2003). Critical roles for Rac1 and Rac2 GTPases in B cell development and signaling. Science 302:459-462.
Wang, Y., Bjes, E. S., and Esser, A. F. (2000). Molecular aspects of complement-mediated bacterial killing. Periplasmic conversion of C9 from a protoxin to a toxin. J. Biol. Chem. 275, 4687-4692.
Wojciak-Stothard, B., Williams, L., and Ridley, A. J. (1999). Monocyte adhesion and spreading on human endothelial cells is dependent on Rho-regulated receptor clustering. J. Cell Biol. 145, 1293-1307.
Yang, D., Chen, Q., Hoover, D. M., Staley, P., Tucker, K. D., Lubkowski, J., and Oppenheim, J. J. (2003). Many chemokines including CCL20/MIP-3alpha display antimicrobial activity. J. Leukoc. Biol. 74, 448-455.

1. An immunogenic composition comprising:
one or more antigens against which an immune response is sought, and
an immunoadjuvant which consists of a Rho GTPase activator able to maintain a Rho GTPase protein in a form bound to GTP;
wherein the said immunoadjuvant is able to enhance the immune response against the said one or more antigens and wherein the said immunoadjuvant is distinct from anyone of the said one or more antigens.

2. The immunogenic composition according to claim 1 wherein the immunoadjuvant is a Rho GTPase activator which is able to activate a Rho GTPase through a post-translational modification.

3. The immunogenic composition according to claim 1 wherein said immunoadjuvant compound is selected from the group consisting of:
a polypeptide comprising the amino acid sequence starting at the amino acid residue 720 and ending at the amino acid residue 1014 of sequence SEQ ID NO:1,
a polypeptide comprising the amino acid sequence starting at the amino acid residue 720 and ending at the amino acid residue 1014 of sequence SEQ ID NO:2,
a polypeptide comprising the amino acid sequence starting at the amino acid residue 720 and ending at the amino acid residue 1014 of sequence SEQ ID NO:3,
a polypeptide comprising the amino acid sequence starting at the amino acid residue 1146 and ending at the amino acid residue 1451 of sequence SEQ ID NO:4,
a polypeptide comprising the amino acid sequence SEQ ID NO:5,
a polypeptide comprising the amino acid sequence SEQ ID NO:6,
a polypeptide comprising the amino acid sequence SEQ ID NO:7,
a polypeptide comprising the amino acid sequence SEQ ID NO:8, and
a polypeptide comprising the amino acid sequence SEQ ID NO:9.

4. The immunogenic composition according to claim 1 wherein said immunoadjuvant compound is selected from the group consisting of:
a polypeptide comprising the amino acid sequence SEQ ID NO:1,
a polypeptide comprising the amino acid sequence SEQ ID NO:2,
a polypeptide comprising the amino acid sequence SEQ ID NO:3, and
a polypeptide comprising the amino acid sequence SEQ ID NO:4.

5. The immunogenic composition according to claim 1, wherein said immunoadjuvant compound is a protein comprising a polypeptide consisting of; from the N-terminal end to the C-terminal end, respectively:
a) the injection domain of a Rho GTPase activator, and
b) the catalytic domain of a Rho GTPase activator.

6. The immunogenic composition according to claim 5, wherein said injection domain of a Rho GTPase activator is a polypeptide selected from the group consisting of:
a polypeptide comprising the amino acid sequence starting at the amino acid residue 1 and ending at the amino acid residue 719 of sequence SEQ ID NO:1;
a polypeptide comprising the amino acid sequence starting at the amino acid residue 1 and ending at the amino acid residue 719 of sequence SEQ ID NO:2;
a polypeptide comprising the amino acid sequence starting at the amino acid residue 1 and ending at the amino acid residue 719 of sequence SEQ ID NO:3; and
a polypeptide comprising the amino acid sequence starting at the amino acid residue 1 and ending at the amino acid residue 1145 of sequence SEQ ID NO:4.

7. The immunogenic composition according to claim 5, wherein said catalytic domain of a Rho GTPase activator is a polypeptide selected from the group consisting of:
a polypeptide comprising the amino acid sequence starting at the amino acid residue 720 and ending at the amino acid residue 1014 of sequence SEQ ID NO:1,
a polypeptide comprising the amino acid sequence starting at the amino acid residue 720 and ending at the amino acid residue 1014 of sequence SEQ ID NO:2,
a polypeptide comprising the amino acid sequence starting at the amino acid residue 720 and ending at the amino acid residue 1014 of sequence SEQ ID NO:3,
a polypeptide comprising the amino acid sequence starting at the amino acid residue 1146 and ending at the amino acid residue 1451 of sequence SEQ ID NO:4,
a polypeptide comprising the amino acid sequence SEQ ID NO:5,
a polypeptide comprising the amino acid sequence SEQ ID NO:6,
a polypeptide comprising the amino acid sequence SEQ ID NO:7,
a polypeptide comprising the amino acid sequence SEQ ID NO:8, and
a polypeptide comprising the amino acid sequence SEQ ID NO:9.

8. The immunogenic composition according to claim 1, wherein the one or more antigens are selected from the group consisting of a hormone, a protein, a drug, an enzyme, a vaccine composition against bacterial, viral, fungal, prion, or parasitic infections, a component produced by microorganisms, inactivated bacterial toxins such as cholera toxin, ST and LT from Escherichia coli, tetanus toxin from Clostridium tetani, and proteins derived from HIV viruses.

9. The immunogenic composition according to claim 1 for administration to a mucosal surface.

10. The immunogenic composition according to claim 1, for an oral administration.

11. The immunogenic composition according to claim 1, wherein the said immunogenic composition consists of a vaccine composition.

12. The immunogenic composition according to claim 11 for administration to a mucosal surface.

13. The immunogenic composition according to claim 11 for oral composition.

14. A protein comprising a polypeptide consisting of; from the N-terminal end to the C-terminal end, respectively:
a) the injection domain of a Rho GTPase activator according to claim 5, and
b) the catalytic domain of a Rho GTPase activator according to claim 5.

15. A method for inducing an immune response in a patient in need thereof, the said method comprising the steps of:
(i) providing an immunogenic composition as defined in claim 1 and
(ii) administering the immunogenic composition provided in step (i) to a patient in need thereof.

16. The method for inducing an immune response in a patient in need thereof according to claim 15 wherein in step (ii) the said immunogenic composition is administered by mucosal route to the patient in need.

17. The method for inducing an immune response in a patient in need thereof according to claim 15 wherein in step (ii) the said immunogenic composition is administered by oral route to the patient in need.

18. A method for preparing an immunogenic composition able to induce an immune response to one or more antigens, the said method comprising the steps of:
(iv) providing one or more antigens against which an immune response is sought
(v) providing an immunoadjuvant as defined in claim 1 and
(vi) mixing the said one or more antigens from step (i) and the immunoadjuvant from step (ii) optionally in the presence of one or more pharmaceutically acceptable excipients.
